SEQUENCE LISTING (85 sequences)

SEQ ID NO: 1 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 2 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 3 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttgtgttaatgctatttgcttgtgttgtatggttaaaattggtgtcttatgcacatacaaactatgatatgagagcacttaccaaatcagttgaaaagggagaagctctgcccgatactctgaacatggagtatccttacaatgtaaccttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttctcattcagtattcttggtcaacctatgtgcgtactgctatattaccatgacttaatgaataggaaaggcaaacttgactga

SEQ ID NO: 4 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVVLMLFACVVWLKLVSYAHTNYDMRALTKSVEKGEALPDTLNMEYPYNVTFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFSFSILGQPMCVLLYYHDLMNRKGKLD

SEQ ID NO: 5 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaactcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaatttccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 6 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaactcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattagcttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgtcggtcaacctatgtgcgtactgctatattaccatgacttaatgaataggaaaggcaaacttgactga

SEQ ID NO: 7 - protein, 498 aa, Glycine max
MAISDEPETVATALNHSSLRRRPTAAGLFNSPETTTDSSGDDLAKDSGSDDSISSDAANSQPQQKQDTDFSVLKLAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLISFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 8 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccgaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttttgcacatacaaactatgatatgagagcacttaccaaatcagttgaaaagggagaagctctgcccgatactctgaacatggaatatccttacaatgtaagcttcaagagcttagcatatttcctgcttgcccctacattatgttaccagccaagctatcctcgcacaccttatattcgaaagggttggctgtttcgccaacttctcaagctggtaatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatagtggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 9 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccgaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttttgcacatacaaactatgatatgagagcacttaccaaatccgttgaaaagggagaagctctgcccgatactctgaacatggaatatccttacaatgtaagcttcaagagcttagcatatttcctgttagcccctacattatgttaccagccaagctatcctcgcacaccttatattcgaaagggttggctgtttcgccaacttttgaagctggtcatctttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatagttgcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgtcggtcaacctatgtgcgtactgctatattaccatgacttaatgaataggaaaggcaaacttgactga

SEQ ID NO: 10 - protein, 498 aa, Glycine max
MAISDEPETVATALNHSSLRRRPTAAGLFNSPETTTDSSGDDLAEDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSFAHTNYDMRALTKSVEKGEALPDTLNMEYPYNVSFKSLAYFLLAPTLCYQPSYPRTPYIRKGWLFRQLLKLVIFTGVMGFIIEQYINPIVQNSQHPLKGNLLYAIERVLKLSVPNLYVWLCMFYCFFHLWLNIVAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 11 - DNA, 1497 bp, Glycine max
atggcgatttccgatgagcctgaaactgtagccactgctctcaacctctcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagcgcaaagtcattgagagactggcccctcttcatgtgttgtctttctcttgtggtatttccttttgctgcatttatggtggagaagttggcacagcagaagtgtatacccgaaccagttgttgttgtacttcatataatcattacctcagcttcacttttctatccagttttagtaattctcaagtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 12 - DNA, 1497 bp, Glycine max
atggcgatttccgatgagcctgaaactgtagccactgctctcaacctctcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagcgcaaagtcattgagagactggcccctcttcatgtgttgtctttctcttgtggtatttccttttgctgcatttatggtggagaagttggcacagcagaagtgtatacccgaaccagttgttgttgtacttcatataatcattacctcagcttcacttttctatccagttttagtaattctcaaatgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgtcggtcaacctatgtgcgtactgctatattaccatgacttaatgaataggaaaggcaaacttgactga

SEQ ID NO: 13 - protein, 498 aa, Glycine max
MAISDEPETVATALNLSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSAKSLRDWPLFMCCLSLVVFPFAAFMVEKLAQQKCIPEPVVVVLHIIITSASLFYPVLVILKCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 14 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaatttccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 15 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaatttctttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgtaggtcaacctatgtgcgtactgctatattaccatgacttaatgaataggaaaggcaaacttgactga

SEQ ID NO: 16 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLISFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 17 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 18 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgtaggtcaacctatgtgcgtactgctatattaccatgacttaatgaataggaaaggcaaacttgactga

SEQ ID NO: 19 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 20 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 21 - DNA, 1497 bp, Glycine max; identical to SEQ ID NO: 20

SEQ ID NO: 22 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 23 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 24 - DNA, 1497 bp, Glycine max; identical to SEQ ID NO: 23

SEQ ID NO: 25 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 26 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagcgcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 27 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagcgcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgtcggtcaacctatgtgcgtactgctatattaccatgacttaatgaataggaaaggcaaacttgactga

SEQ ID NO: 28 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSAKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 29 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaatttctttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 30 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLISFLVSALFHELCIAVPCHIFKLWAFGGIMFQVPLVFITNYLQNKFRNSMVGNMIFWFIFSILGQPMCVLLYYHDLMNRKGKLD

SEQ ID NO: 31 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaactcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaatttctttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 32 - protein, 498 aa, Glycine max
MAISDEPETVATALNHSSLRRRPTAAGLFNSPETTTDSSGDDLAKDSGSDDSISSDAANSQPQQKQDTDFSVLKLAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLISFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 33 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcttcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaatttctttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattgttggtcaacctatgtgcgtactgctatattaccatgacttaatgaataggaaaggcaaacttgactga

SEQ ID NO: 34 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLISFLVSALFHELCIAVPCHIFKLWAFGGIMFQVPLVFITNYLQNKFRNSMVGNMIFWFIFSIVGQPMCVLLYYHDLMNRKGKLD

SEQ ID NO: 35 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcaacaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 36 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLNNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 37 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcaaaaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 38 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLKNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 39 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcgacaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 40 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLDNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 41 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctccttaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 42 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLLNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 43 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcgccaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 44 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLANLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 45 - DNA, 1497 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctctggaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 46 - protein, 498 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLWNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 47 - DNA, 1494 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcagagtcacgcgggcctcaacctctgtatagtagtccttgttgctgtgaatagccgactcatcattgagaatttaatgaagtatggttggttgatcaaatctggcttttggtttagctcaaagtcattgagagactggcccctcttcatgtgttgtctttctcttgtggtatttccttttgctgcatttatagtggagaagttggcacagcagaagtgtatacccgaaccagttgttgttgtacttcatataatcattacctcagcttcacttttctatccagttttagtaattctcaggtgtgattctgcttttctatcaggtgttacgttaatgctatttgcttgtgttgtatggttaaaattggtgtcttatgcacatacaaactatgatatgagagcacttaccaaatcagttgaaaagggagaagctctgcccgatactctgaacatggactatccttacaatgtaagcttcaagagcttagcatatttcctggttgcccctacattatgttaccagccaagctatcctcgcacaccttatattcgaaagggttggctgtttcgccaacttgtcaagctgataatatttacaggagttatgggatttataatagaacaatacattaatcccattgtacaaaattcacagcatcctctcaagggaaaccttctttacgccatcgagagagttctgaagctttctgttccaaatttatatgtgtggctctgcatgttctattgctttttccacctttggttaaatatattggcagagcttcttcgatttggtgatcgtgaattctaccaggattggtggaatgccaaaactgttgaagattattggaggatgtggaatatgcctgttcacaaatggatgatccgccacctatattttccatgtttaaggcacggtataccaaaggccgttgctcttttaattgccttcctggtttctgctttattccatgagctgtgcatcgctgttccttgccacatattcaagttgtgggctttcggtggaattatgtttcaggttcctttggtcttcatcactaattatctgcaaaataaattcagaaactcgatggttggaaatatgattttttggttcatattcagtattcttggtcaacctatgtgcgtactgctatattaccatgacttaatgaataggaaaggcaaacttgactga

SEQ ID NO: 48 - protein, 497 aa, Glycine max
MAISDEPETVATALNHSSLRRRPTAAGLFNSPETTTDSSGDDLAKDSGSDDSISSDAANSQPQQKQDTDFSVLKFAYRPSVPAHRKVKESPLSSDTIFRQSHAGLNLCIVVLVAVNSRLIIENLMKYGWLIKSGFWFSSKSLRDWPLFMCCLSLVVFPFAAFIVEKLAQQKCIPEPVVVVLHIIITSASLFYPVLVILRCDSAFLSGVTLMLFACVVWLKLVSYAHTNYDMRALTKSVEKGEALPDTLNMDYPYNVSFKSLAYFLVAPTLCYQPSYPRTPYIRKGWLFRQLVKLIIFTGVMGFIIEQYINPIVQNSQHPLKGNLLYAIERVLKLSVPNLYVWLCMFYCFFHLWLNILAELLRFGDREFYQDWWNAKTVEDYWRMWNMPVHKWMIRHLYFPCLRHGIPKAVALLIAFLVSALFHELCIAVPCHIFKLWAFGGIMFQVPLVFITNYLQNKFRNSMVGNMIFWFIFSILGQPMCVLLYYHDLMNRKGKLD

SEQ ID NO: 49 - DNA, 1515 bp, Glycine max
atggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacagcgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcttcaacctttgtatagtagtccttgttgctgtgaatagccgactcatcattgagaatttaatgaagtatggttggttgatcaaatctggcttttggtttagttcaaagtcattgagagactggccccttttcatgtgttgtctttctcttgtggtatttcctttcgctgcctttatagtggagaagttggcacaacggaagtgtatacccgaaccagttgttgttgtacttcatataatcattacctcaacttcgcttttctatccagttttagttattctcaggtgtgattctgcttttgtatcaggtgtcacgttaatgctgttttcttgtgttgtatggttaaaattggtgtcttatgcacatacaaactatgatatgagagcacttaccaaattagttgaaaagggagaagcactgctcgatactctgaacatggactatccttacaacgtaagcttcaagagcttggcatatttcctggttgcccctacattatgttaccagccaagctatcctcgcacaccttatattcgaaagggttggttgtttcgccaacttgtcaagctgataatatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcctggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaattgccttcctggtttctgctttattccatgagctgtgcattgctgttccttgccacatattcaagttgtgggctttcggtggaattatgtttcaggttcctttggtcttgatcactaattatctgcaaaataaattcagaaactcaatggttggaaatatgattttttggttcatcttcagtatccttggtcaacctatgtgtgtactgctatactaccatgacttgatgaataggaaaggcaaacttgactga

SEQ ID NO: 50 - protein, 504 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLFNLCIVVLVAVNSRLIIENLMKYGWLIKSGFWFSSKSLRDWPLFMCCLSLVVFPFAAFIVEKLAQRKCIPEPVVVVLHIIITSTSLFYPVLVILRCDSAFVSGVTLMLFSCVVWLKLVSYAHTNYDMRALTKLVEKGEALLDTLNMDYPYNVSFKSLAYFLVAPTLCYQPSYPRTPYIRKGWLFRQLVKLIIFTGVMGFIIEQYINPIVQNSQHPLKGNLLYATERVLKLSVPNLYVWLCMFYCFFHLWLNILAELLRFGDREFYKDWWNAKTVEDYWRMWNMPVHKWMIRHLYFPCLRHGLPKAAALLIAFLVSALFHELCIAVPCHIFKLWAFGGIMFQVPLVLITNYLQNKFRNSMVGNMIFWFIFSILGQPMCVLLYYHDLMNRKGKLD

SEQ ID NO: 51 - DNA, 1515 bp, Glycine max
atggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacagcgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcagtaacctttgtatagtagtccttgttgctgtgaatagccgactcatcattgagaatttaatgaagtatggttggttgatcaaatctggcttttggtttagttcaaagtcattgagagactggccccttttcatgtgttgtctttctcttgtggtatttcctttcgctgcctttatagtggagaagttggcacaacggaagtgtatacccgaaccagttgttgttgtacttcatataatcattacctcaacttcgcttttctatccagttttagttattctcaggtgtgattctgcttttgtatcaggtgtcacgttaatgctgttttcttgtgttgtatggttaaaattggtgtcttatgcacatacaaactatgatatgagagcacttaccaaattagttgaaaagggagaagcactgctcgatactctgaacatggactatccttacaacgtaagcttcaagagcttggcatatttcctggttgcccctacattatgttaccagccaagctatcctcgcacaccttatattcgaaagggttggttgtttcgccaacttgtcaagctgataatatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcctggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaattgccttcctggtttctgctttattccatgagctgtgcattgctgttccttgccacatattcaagttgtgggctttcggtggaattatgtttcaggttcctttggtcttgatcactaattatctgcaaaataaattcagaaactcaatggttggaaatatgattttttggttcatcttcagtatccttggtcaacctatgtgtgtactgctatactaccatgacttgatgaataggaaaggcaaacttgactga

SEQ ID NO: 52 - protein, 504 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQRKCIPEPVVVVLHIIITSTSLFYPVLVILRCDSAFVSGVTLMLFSCVVWLKLVSYAHTNYDMRALTKLVEKGEALLDTLNMDYPYNVSFKSLAYFLVAPTLCYQPSYPRTPYIRKGWLFRQLVKLIIFTGVMGFIIEQYINPIVQNSQHPLKGNLLYATERVLKLSVPNLYVWLCMFYCFFHLWLNILAELLRFGDREFYKDWWNAKTVEDYWRMWNMPVHKWMIRHLYFPCLRHGLPKAAALLIAFLVSALFHELCIAVPCHIFKLWAFGGIMFQVPLVLITNYLQNKFRNSMVGNMIFWFIFSILGQPMCVLLYYHDLMNRKGKLD

SEQ ID NO: 53 - DNA, 1515 bp, Glycine max
atggcgatttccgatgagcctgaaagtgtagccactgctctcaaccactcttccctgcgccgccgtccctccgccacctccaccgccggcctcttcaattcgcctgagacaaccaccgacagttccggtgatgacttggccaaggattctggttccgacgactccatcaacagcgacgacgccgccgtcaattcccaacagcaaaacgaaaaacaagacactgatttctccgtcctcaaattcgcctaccgtccttccgtccccgctcaccgcaaagtgaaggaaagtccgctcagctccgacactattttccgtcagagtcacgcgggcctcagtaacctttgtatagtagtccttgttgctgtgaatagccgactcatcattgagaatttaatgaagtatggttggttgatcaaatctggcttttggtttagttcaaagtcattgagagactggccccttttcatgtgttgtctttctcttgtggtatttcctttcgctgcctttatagtggagaagttggcacaacggaagtgtatacccgaaccagttgttgttgtacttcatataatcattacctcaacttcgcttttctatccagttttagttattctcaggtgtgattctgcttttgtatcaggtgtcacgttaatgctgttttcttgtgttgtatggttaaaattggtgtcttatgcacatacaaactatgatatgagagcacttaccaaattagttgaaaagggagaagcactgctcgatactctgaacatggactatccttacaacgtaagcttcaagagcttggcatatttcctggttgcccctacattatgttaccagccaagctatcctcgcacaccttatattcgaaagggttggttgtttcgccaacttgtcaagctgataatatttacaggagttatgggatttataatagaacaatatattaatcccatagtacaaaattcacagcatcctctcaagggaaaccttctttacgccaccgagagagttctgaagctttctgttccaaatttatatgtgtggctctgcatgttctattgctttttccacctttggttaaatatcctggcagagcttcttcgatttggtgatcgtgaattctacaaggattggtggaatgccaaaactgtcgaagattattggaggatgtggaatatgcctgttcacaaatggatgatccgccacctatattttccatgtttaaggcacggtctaccaaaggctgctgctcttttaattgccttcctggtttctgctttattccatgagctgtgcattgctgttccttgccacatattcaagttgtgggctttcggtggaattatgtttcaggttcctttggtcttgatcactaattatctgcaaaataaattcagaaactcaatggttggaaatatgattttttggttcatcttcagtatcgttggtcaacctatgtgtgtactgctatactaccatgacttgatgaataggaaaggcaaacttgactga

SEQ ID NO: 54 - protein, 504 aa, Glycine max
MAISDEPESVATALNHSSLRRRPSATSTAGLFNSPETTTDSSGDDLAKDSGSDDSINSDDAAVNSQQQNEKQDTDFSVLKFAYRPSVPAHRKVKESPLSSDTIFRQSHAGLSNLCIVVLVAVNSRLIIENLMKYGWLIKSGFWFSSKSLRDWPLFMCCLSLVVFPFAAFIVEKLAQRKCIPEPVVVVLHIIITSTSLFYPVLVILRCDSAFVSGVTLMLFSCVVWLKLVSYAHTNYDMRALTKLVEKGEALLDTLNMDYPYNVSFKSLAYFLVAPTLCYQPSYPRTPYIRKGWLFRQLVKLIIFTGVMGFIIEQYINPIVQNSQHPLKGNLLYATERVLKLSVPNLYVWLCMFYCFFHLWLNILAELLRFGDREFYKDWWNAKTVEDYWRMWNMPVHKWMIRHLYFPCLRHGLPKAAALLIAFLVSALFHELCIAVPCHIFKLWAFGGIMFQVPLVLITNYLQNKFRNSMVGNMIFWFIFSIVGQPMCVLLYYHDLMNRKGKLD

SEQ ID NO: 55 - DNA, 21 bp, Artificial Sequence
  DGAT1 flanking forward primer
ttagtttagtagaacctcgtg

SEQ ID NO: 56 - DNA, 21 bp, Artificial Sequence
  DGAT1 flainking reverse primer
aacatagaaatatcgaatggg

SEQ ID NO: 57 - DNA, 19 bp, Artificial Sequence
  DGAT1 internal gene specific primer
tgacttggccaaggattcc

SEQ ID NO: 58 - protein, 18 aa, Glycine max
LRRRPSATSTAGLFNSPE

SEQ ID NO: 59 - DNA, 20 bp, Artificial Sequence
  DGAT1a gRNA
gtactatacagaggttgaag

SEQ ID NO: 60 - DNA, 7812 bp, Glycine max
atggcgatttccgatgagcctgaaactgtagccactgctctcaaccactcttccctgcgccgccgtcccaccgccgctggcctcttcaattcgcccgagacgaccaccgacagttccggtgatgacttggccaaggattccggttccgacgactccatcagcagcgacgccgccaattcgcaaccgcaacaaaaacaagacactgatttctccgtcctcaaattcgcctaccgtccttccgtccccgctcatcgcaaagtgaaggaaagtccgctcagctccgacaccattttccgtcaggttctcgccgttaacttctactttacaattattacaattccgtaactatttcaaactcaaccagaacggtaccgtttcgttcccgttctgcattaataaactactcaatctcaatgcatgactcatgaatgcaagcgactcgtagtcttttaggtgcttaaaatagtgtatgctactgctagttagttaatttgtgtgtacgggaaactagttatcaatttatgatttgaatttaatacaattgaattggtggatcaggtttttgtacctgctattgtgtttggtttataattaggtgtgatgagtctgatatgtaattatatttctttactaattattcaatcagttgcagagtcacgcgggcctcttcaacctctgtatagtagtccttgttgctgtgaatagccgactcatcattgagaatttaatgaaggttttgcctcttccatgtttaattgtgcattcacctgcaacaattttctgttctttttatttatgaccttgattttgacggttcgggttttgttgtctgctttaatgagtgttttatttgttctgcagtatggttggttgatcaaatctggcttttggtttagctcaaagtcattgagagactggcccctcttcatgtgttggtaatgagtgtaatgaattccctaaagaattaactatgtactatgtaattattctgaaatgttgttcaacagtatgtggttgtgcacttggtttgcagtctttctcttgtggtatttccttttgctgcatttatagtggagaagttggcacagcagaagtgtatacccgaaccagttaagtgatcaggccatatgagcttttattagatttcattcgttttataacctaaaatatttttgggatatggttagttgtgatgtgctattcttaactgttcaatctttaatatttgcatacatgagaatattatattactttaatattgcaggttgttgttgtacttcatataatcattacctcagcttcacttttctatccagttttagtaattctcaggtcagttaagttcttttgtatatattctggggctgcagttgaggcaccttttgttttgacatgggtggttacacaatgtgtccattttgaaagaatttggtaatagaatatggttttaagttgatagtggtgttaagtaacaaactttgtcactatcctgcacaggaatttaaatttttttattatattttaaaattacttgagaagaaatttaaagcatttgggtgaaccttagtttaatgtttggttcttatcatgttggaattgaattatgttgcaaaatatgaagatgaatgctgagggatgaaataattatacagttgtgcattcttagtcatttcatagataaggatatcccagaaagatgattactgacttgtaacattctttgcaggtgtgattctgcttttctatcaggtgttacgttaatgctatttgcttgtgttgtatggttaaaattggtgtcttatgcacatacaaactatgatatgagagcacttaccaaatcagttgaaaaggtaataaatactctctgtttaaacggagagaaaattttgaatttttttttcttaaaagtcaacttttctctcttgtctttaactaaacaaaagaaaatctatctttacgtgttttttctttcatctaattttttccttaaccaaacatattcttaaattctacttggcacttgtaagtgggtagataactatcacaaaatatcatcgtcacttatttgtatactttaaagatgaccatatgttgtacaatgtttgtgatgattagtttttgaacctgaacatcctgcttgacttagcagtagcttatggatcatgtactcttaaaacttcattatatgctgaagtacagttttatttcatcttcatttgaattacttccatcctgtgttccgctttattgcttccttctatctagatggttagctcttcaaacgtgtgaatgtgtgatagataaaccatctgctttccctccttattcaaacatgtttcatgtgttatagggagaagctctgcccgatactctgaacatggactatccttacaatgtaagcttcaagagcttagcatatttcctggttgcccctacattatgttaccaggtagcagtactttcaagtgatttagttaatttttggaagcaattttttttaattgttgtggtgttgcctcatttagattcgcctattttacatttgcattcacttttctagtcttagattgcttataggcacaaccatgtggagttgcttactcatatatctttggtgtggaatatgtctgaggtttttagtcagtgtctaattggacatgctgttgggaacccagaaacagagtatgagaaaagaatgcttcttgtactaattacagaatattgaatgacaacaaagaaaaggggaaaattttgctcaaagagtttctcctttcacaacacagatgcttaattgaatgactcccccccccaaaaaaaaaaaaaaatcttcctatatgtttctcactaaccccctctaactaagaatcaaactaattgctctaaccatcccaactaatttcactgctaggaattaagttcccatctaacaagtataattgatccagaatgttgggttggctacatagtaggatttctctgaatgactctttaatcattgtcttcaatacattctatacctttcagcaaatttctccatttattatcctttctttcctttccatcttctaattttggccatttgacctcgttcatctcacattcaatactcctttctattttgtagcatagaatattgtgtaatgaaatctatagcaggaatcccccaaacccttctcatgccccccctttacttccttcgtacaaaattcccattgcaatctgctagtttctagacacaacctgctagtcaatgcacggtgtaggagccacaatctgttagtctatgtgttcctcaatctgcatataatacttcttgcttcttgcttttatatgaagctaagaataagattttaagggaatgagtattacatgactccccggcataaaatgtagcatgcatttctaatgctttgtgagaatactggattggctttggcatcttcagtgtttacctacgttgcagaaattttgcttggggttctttaagtaggtttctgcagttcatttgtctttgcattttcctgcataaatgtatctccatgtgaattgcatttgttattttttttattctgagagcaaggtctttcacatttttatttgcatgtattttgttttatgaactgctcttttagccaagctatcctcgcacaccttatattcgaaagggttggctgtttcgccaacttgtcaagctgataatatttacaggagttatgggatttataatagaacaagtaagcagttcctgttaataaattgtgttcatttctacctttttttcatttctttttttatgtacatttttttcttgaggtctatttcaccaattctactggtattctgacattattttgactgtaattttgcagtacattaatcccattgtacaaaattcacagcatcctctcaagggaaaccttctttacgccatcgagagagttctgaagctttctgttccaaatttatatgtgtggctctgcatgttctattgctttttccacctttggtattccatctcttgtttggttcaacatgtgtgtatgtgtgtgcattttccttacaagatattagtatgaagttatcttcacctgccataaattgaataattatagttctagtttattttgtttgatgccttgacttttgtaccctggcaattttctgatgatgatgatgttataagttagtaattgattaatctttcaaatcatccttgaagtgatatccaccttcctaaacaccttaaaatagtaattacatcatcaatgggaagatctagctgatctaaaggtgtgcatgtagagactattggtgtcttgagtgtacacttctaaagatataaaataaactttatagcctgtttggtgtgccaccaatgattaatttaagtgacactctgacggatatgtgaaaggatttgagagccttattttattgatggatttgaatatttttcctttggccagtgaggatatattattcttcatttatgctcttttttctcctctttaagaactactcttagaaaaaaaactagatctttttttgcaagcataaatgctgatctgcttgtctcatttgtagatattagaaaaggttcacctttgattttttaagatttagttaatatttgaggaagcttatcattggagtgactgtcgtaatgtaggttaaatatattggcagagcttcttcgatttggtgatcgtgaattctaccaggattggtggaatgccaaaactgttgaagatgtaagttatttgtcctgatattttgtcgtttacttacagtattttcagtcttcataaagagaaattgtgactttttgtttttgtttctgtatttgtgctgttgttgctgcttccatcagtattggaggatgtggaatatggtatgtctctttttcagacttaattttgatgaacaaattgattttcttgtgttcagcaaaatgattttcaggtctcctgttgggatgaaattcatgtgaacacacgtacatatatttgatataactatccctttaaatgtataatgagggttgacaactctccaagactattttaacaacggaagaaagagaagttacaacttttttccatcttactgactaaatggttttaaaactgtctttcagaacatatcattaattatttatcacgtaaagttccaaaccattagtataatctgagcggaatcttttacattgcagcctgttcacaaatggatgatccgccacctatattttccatgtttaaggcacggtataccaaaggtaatcaagcatccttctgcgttgctgaatggatcctgaacttatttggtactgtaaaacatttttaggatttgtcagcctctgtttaccatctcgggttgctactaagatcatcacatttaacatagttgaattaaaatgaacatgttacttttatcctaataatcacacttatgtaaaaacttatcaataaacctattacataagttttttttcaaaaaataagtatttgcactaattgtatggtttgatatgtttggtgcactgggggagctatatgtagaaagtttgcaattggtagacagtaatttagcttcatcttctgttctgtcatttcagataaattactttggagtgcataaacatgactaaaacaagaattgcaaaggaactccattaaaaaaaaaaaaatcttatgtaatcatgttcaatagctttgtgatttttttccttggtctttgccatcaacaatttcctattaacatatagttgtttctcattggttaatatttggattggctctttttttgtcaagcattattggtatttcaatttttagttctagataaaatttttatttttcatcattgtacaggccgttgctcttttaattgccttcctggtttctgctttattccatgaggtgtgttgctcatctctctgtacagccttctctcttctttttttggtattatgtcattatggttgttctgcttgagtgtttgtctgcatcctacactgattgaagtcaaactgtatcttgcaattaatatatttttggagtatcctttcctccttttccttgggctgtttggactttgctaagagttagatagaagggaagggaaaattagttacagatggttagagtagttgattagtcagttataggatttggttagatattaattacggggattaagaggaaaggatccattctatctagttgattagtcagttataggatccattctatctagtatcatttgagaattgagcttggctcttttgaaaggagaaatcctttgtgagggggaaacccttggaggagaattatctcttcttttttctgttctagatgataaaactgcccttttatttctttctttgctttgggttcgtaacagactgtgggagaggactgaaaattagatactaagttgtggctgatttgttaaaaattggtataggacccatgctacttgtctttgaactatctacaacccactatggccccagaaaccttaggcagctagctaccaaaatctgtagtaacttgcagcctgtctgtctgtaccttggcaggtgtagatgttacagcttccatatcttatatcttaaatatagactgacataacaattatgacaaactgttatattttctagttgctagttcttgcaatattaattttggcaccaaaatttccctacatgtactcaaggatgcttactttagccatgaataattttttttttcacgtgttgtaattatattttcaatttatcctgtcattttttcctgaaaaaggtacatacacagaataatttgataacttaaggtggcgcttggtttgagtgttttgtgttttcattttcaaagaaaatagaaaataagagtgaaaatatgtttgcctaaaatttagaaaacattttctatgaaaatatttcaaaaacaagcccaaaactgaaaacaaccaataaactttttcagcctttttctgaagaagtgaaaacacgatgtcactttagagtactttcttctcaaaacacgttttctaaatcttgaaaatctgaaaatgaaaataaaaattattttgagaaaatgaaaacagaaaataaacattcaaatcaaacgccaccttaattgtcatagacagttgtgcaatttttctcacgatatctctttcgtctgagtgcagctgtgcatcgctgttccttgccacatattcaagttgtgggctttcggtggaattatgtttcaggtaaaatcaaattaccgcactaaacttttgttttcccatcttctattattaaactaaagcattttaattcagagtaagagtaacacgaattttccaagaaaccaaacttctcccctcttccctcacgaagaaggtgaaggattgccgagatttttttttcctgttcttttatggccaaacgatagaaagtattctctggaataatgaatttgtgccatgtttgaaagttgatgattatgatatcatccatctttttggttcaattaacatgataaggaatgacttttttctaggttcctttggtcttcatcactaattatctgcaaaataaattcagaaactcgatggtacgtctgctatttaagcatattgattcataatatatcttattactccatcatcttgaatttgattcattggttttttaatttgtttttgtcttcatgccaggttggaaatatgattttttggttcatattcagtattcttggtcaacctatgtgcgtactgctatattaccatgacttaatgaataggaaaggcaaacttgactga

SEQ ID NO: 61 - protein, 7 aa, glycine max
APTLCYQ

SEQ ID NO: 62 - protein, 13 aa, glycine max
  misc_feature (8)..(8): x is any amino acid
FGDREFYXDWWNA

SEQ ID NO: 63 - protein, 6 aa, glycine max
LLYYHD

SEQ ID NO: 64 - protein, 44 aa, glycine max
  misc_feature (8)..(8): X is any amino acid
  misc_feature (11)..(11): X is any amino acid
  misc_feature (18)..(18): X is any amino acid
  misc_feature (22)..(22): X is any amino acid
  misc_feature (26)..(26): X is any amino acid
  misc_feature (30)..(30): X is any amino acid
  misc_feature (42)..(42): X is any amino acid
ESPLSSDXIFXQSHAGLXNLCXVVLXAVNXRLIIENLMKYGXLI

SEQ ID NO: 65 - protein, 19 aa, glycine max
  misc_feature (13)..(13): X is any amino acid
  misc_feature (16)..(16): X is any amino acid
GFIIEQYINPIVXNSXHPL

SEQ ID NO: 66 - DNA, 1554 bp, glycine max
atggcgatctccgatgtgcctgcagccgctggcacgaccgccactaccaccagcgactcagatctccgacagccttctctgcggcgcaggtcctccgccggagtcctcttcgacgctgccagagattccggctccgacaattccctgaccggcaaaatcaccgacgacgacaacatcaaagatcacaagccgaataatcacgcagcctccgacgacaatgtgggcgccgccgccaatgacgctgggcaggagcaccgacaaccggtcgccgatttcaaatacgcttaccgtccctccgttcccgcgcaccgcagaatcaaggagagcccccttagctccgacaacatcttcagacagagtcatgcaggactgttcaatctctgcatagtagtgcttgttgccgtgaacagcagacttatcattgagaatttaatgaagtatggttggttgatcaagtatggcttttggtttagttcaaaatcattgagagattggcctctcttcatgtgctgtcttagtcttgccatatttccacttgctgcctttgttgtggaaaggttggcacaacaaaagtgtatttctgaaccagttgttgttctacttcatctaataatatcaactgttgaactgtgctatccggttttagtaatactcaggtgtgattctgcttttgtatctggtgtcacgttgatgctattaacttgcattgtgtggttaaaattggtgtcatatgcacatacaaactatgatatgagagcacttactgtttcgaatgaaaagggagaaacattacccaatactttgattatggagtatccgtacactgtgaccttcaggagtttggcatacttcatggttgctcctacattatgctatcagacaagctatcctcgcacaccttcagttcgaaagggttgggtgtttcgtcaacttgtcaagctgataatatttacaggagttatgggatttataatagaacaatatatgaatcctattgtacaaaactcaactcatcctttgaagggaaaccttctatatgccattgagagaattctgaagctttctgtcccaaatgtatatgtgtggctctgcatgttctactgctttttccacctttggttaaatatacttgcagagcttgttcgatttggtgatcgtgagttctataaagattggtggaatgccaaaactgttgaagagtattggaggatgtggaatatgcctgtgcacaaatggatggttcgccacatatattttccatgcttaaggcgtggtatacccaagggtgctgcttcattaattgcattcctggtttctgctgtgtttcatgagttatgcattgccgttccttgccacatgttcaagttgtgggcttttataggaattatgtttcaggttcctttggtcttgatcactaattacctccaaaataaatacagaaactcaatggttggaaatatgattttttggttcatattttgtattcttggtcaaccaatgagcgtactattgtactaccatgacttgatgaatagaaaaggagaagttgactaa

SEQ ID NO: 67 - protein, 517 aa, glycine max
MAISDVPAAAGTTATTTSDSDLRQPSLRRRSSAGVLFDAARDSGSDNSLTGKITDDDNIKDHKPNNHAASDDNVGAAANDAGQEHRQPVADFKYAYRPSVPAHRRIKESPLSSDNIFRQSHAGLFNLCIVVLVAVNSRLIIENLMKYGWLIKYGFWFSSKSLRDWPLFMCCLSLAIFPLAAFVVERLAQQKCISEPVVVLLHLIISTVELCYPVLVILRCDSAFVSGVTLMLLTCIVWLKLVSYAHTNYDMRALTVSNEKGETLPNTLIMEYPYTVTFRSLAYFMVAPTLCYQTSYPRTPSVRKGWVFRQLVKLIIFTGVMGFIIEQYMNPIVQNSTHPLKGNLLYAIERILKLSVPNVYVWLCMFYCFFHLWLNILAELVRFGDREFYKDWWNAKTVEEYWRMWNMPVHKWMVRHIYFPCLRRGIPKGAASLIAFLVSAVFHELCIAVPCHMFKLWAFIGIMFQVPLVLITNYLQNKYRNSMVGNMIFWFIFCILGQPMSVLLYYHDLMNRKGEVD

SEQ ID NO: 68 - DNA, 1509 bp, Brassica napus
atggcggttttggattctggaggcgtcgctgtgccgacggagaacggcgtcgcggatctcgacaggctccatcgtcgtaaatcgagatcggattcttccaacggactcctccccgatacttccccgtcggacgatgttggagctgcggcggccgaaagggatcgggttgattccgctgccgaggaggaggctcagggaacagcgaatttagctggcggagatgccgaaactagggaatccgccggaggcgatgtaaggtttacgtatcgaccgtcggttccagctcatcggaggacgagggagagtcctctcagctctgacgctatcttcaaacaaagccatgcaggattgttcaacctctgtgtagttgttcttgttgctgttaacagtagactcatcatcgaaaacctcatgaagtatggttggttgatcagaactgatttttggtttagttctacatccttacgagactggccgcttttcatgtgttgtctttcactttcggtctttcctttggctgccttcacggtcgagaaaatggtacttcagagattcatatctgagcctgttgccatcattcttcatgttattataaccttgacagaggtcttgtatccagtctacgtcacactgaggtgtgattctgccttcttgtcaggtgtcacgttgatgctgctcacttgcattgtgtggctgaagttggtttcttacgctcatactagctacgacataagaaccctagctaattcagctgataaggtcgatcctgaaatctcctaccatgttagcttgaagagcttggcgtatttcatggttgctcctacactgtgttatcagccaagctatccacgttccccatgtatacggaagggttgggtggctcgtcaatttgcgaaactggtcatattcactggactcatgggatttataatagagcagtatataaatcctattgtaaggaactcaaagcatccgttgaaaggggatcttctatacgctattgaaagagtgttgaagctttcagttccaaatctatatgtgtggctctgcatgttctactgcttcttccacctttggttaaacatattggcagagctgctctgcttcggggaccgtgaattctacaaagattggtggaatgcaaaaagcgttggagattattggagaatgtggaatatgcctgttcataaatggatggttcgacatgtatactttccgtgcctgcgcatcaagataccaaaagtacccgccattatcattgctttcttagtctctgcagtctttcatgagttatgcatcgcagttccttgccgtctcttcaatctatgggctttcatgggaattatgtttcaggtccctttggtctttatcacaaactttttacaagaaaggtttggctccatggtgggaaacatgatcttctggttcagcttctgcattttcggacaacccatgtgtgttcttctttattaccatgacctgatgaaccgcaaaggatccatgtcctga

SEQ ID NO: 69 - protein, 503 aa, Brassica napus
MAILDSGGVAVPPTENGVADLDRLHRRKSSSDSSNGLLSDTSPSDDVGAAAAERDRVDSAAEEEAQGTANLAGGDAETRESAGGDVRFTYRPSVPAHRRTRESPLSSDAIFKQSHAGLFNLCVVVLVAVNSRLIIENLMKYGWLIRTDFWFSSTSLRDWPLFMCCLSLSVFPLAAFTVEKMVLQKFISEPVAIILHVIITMTEVLYPVYVTLRCDSAFLSGVTLMLLTCIVWLKLVSYAHTSYDIRTLANSADKVDPEISYYVSLKSLAYFMVAPTLCYQPSYPRSPCIRKGWVARQLAKLVIFTGLMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHVYFPCLRIKIPKVPAIIIAFLVSAVFHELCIAVPCRLFNLWAFMGIMFQVPLVFITNFLQERFGSMVGNMIFGSASCIFGQPMCGLLYYHDLMNRKGSMS

SEQ ID NO: 70 - DNA, 1509 bp, Gossypium hirsutum
atggcgatgtttgagtcaccggagatttcagggagtagtacggcgacggtgatcggtacctcacgtagcgagtcagatcttaatcattttgcgccccgtcgtcgagccgtgaataacgccgtcgatgcagggactagagttgtggagcgtaacaattccggtaatggcgagacagtggacgctagggatcgaatggaatcggctaatttctcgagggaaaacgtgaatgagaatccgactaattcagatacgaggttcacgtatcggccttctgttcctgctcattggaggatcaaagaaagccctctcagctctgacaatatcttccaacagagtcatgcaggcctgtttaacctatgtgttgtagtgcttgttgctgtaaacagccggcttattattgagaacctgatgaagtatgggtggttgatcagaactggcttttggtttagttcgagatcattgagggattggcctctttttatgtgctgtctttctctcccaatattcccaatcgcggcctttgtagttgaaaagttgttgcaacagaatcaaatatctgaacgaactcttattttacttcatatactgattagcacgcttgcagttctatatccagttgttgttattctcaggtgtgattctgcattcttatcaggcattgcattgatgctatttgcttgcattgtctggttaaaattggtatcctatgctcatactaacagtgatatgagatcagttgcgaagtcgactgaaaagggaagtgaaggctgcatgtacaatgttagcttcaggagtctggcatacttcatggcagctcccacattatgttaccagacaagctatcctcgtactgcatcaattagaaaaaattgggtggttcgtcaatttatcaagttaataatatttactggactcatgggtttcataatagaacagtatatcaatccaattgttcagaactctcagcaccctttgaaggcgaactttttatatgccatagaaagaattttgaagctttcagttccaaatacatatgtctggctttgcatgttctacagcttctttcatctctggttaaatatactggctgagcttcttcgttttggggaccgcgagttttataaagattggtggaatgcaaaaactgttgaggagtattggagaatgtggaatatgcctgttcataaatggatggttcgccatatctatttgccatgcttaaggaacggtataccaaagggagttgccattcttatcgccttcttggtttctgctatatttcatgagctctgcattgctgttccttgccacttatttaagttatgggctttctttggcatcatgtttcaggctcccttggtcttgatcactagttatcttcaaaataagttccagagttcaatggtgggaaatatgatattctggttcatattctgcattcttggtcaaccaacgtgcgtacttttatattatcatgatttgatgaatcgcaaaggatcggcagattaa

SEQ ID NO: 71 - protein, 502 aa, Gossypium hirsutum
MAMFESPEISGSSTATVIGTSRSESDLNHFAPRRRAVNNAVDAGTRVVERNNSGNGETVDARDRMESANFSRENVNENPTNSDTRFTYRPSVPAHWRIKESPLSSDNIFQQSHAGLFNLCVVVLVAVNSRLIIENLMKYGWLIRTGFWFSSRSLRDWPLFMCCLSLPIFPIAAFVVEKLLQQNQISERTLILLHILISTLAVLYPVVVILRCDSAFLSGIALMLFACIVWLKLVSYAHTNSDMRSVAKSTEKGSEGCMYNVSFRSLAYFMAAPTLCYQTSYPRTASIRKNWVVRQFIKLIIFTGLMGFIIEQYINPIVQNSQHPLKANFLYAIERILKLSVPNTYVWLCMFYSFFHLWLNILAELLRFGDREFYKDWWNAKTVEEYWRMWNMPVHKWMVRHIYLPCLRNGIPKGVAILIAFLVSAIFHELCIAVPCHLFKLWAFFGIMFQAPLVLITSYLQNKFQSSMVGNMIFWFIFCILGQPTCVLLYYHDLMNRKGSAD

SEQ ID NO: 72 - DNA, 1554 bp, Helianthus annuus
atggcgttactagatacgtcagatatcggagactccaccgccatacgcggcgagataagacggcggaggagcgtgaagcctgatgccggattcggaatcggagacggtttgtatgattcttcatcgtcttctcgaacgaactcgtctgaagaagaaggtgagagtttgactaacggtttcgatgaaaatgaacggatccgagctggtgatgaaactcaaacaacacaggaaaataaacagaagacagatcagagaagagataaaacgagtctgttgcaatatgcgtatcgtgcttcttctccagcgcatcgtagaattaaagagtctccgcttagctccgatgctatcttcaagcagagtcatgcaggcctttttaacctttgcatagtggtgcttgttgctgtaaatggtaggctcattattgagaatctgatgaagtacggattattgatcaattccaatttttggttcagttcaagatcattaagagattggccccttctgatgtgctgcgtctctcttctgttcttccctcttgctgcttacattgttgagaaattggcatggaaaaaacgtatatcagaccctgttgtaatcactctccatgttatagtaactacaactgcgattttatatccggttttcatgattctgagggttgattcagttgttctatcaggtgtttcattgatgctgtgtgcttgcatcaattggttaaaattgacatcttttgtgcatactagttatgacatgcggtcccttgtgaattcaaccgataagggagagacagagtccgagtctttagatatagagttattttatgatgctgacttcaaaagcttggtttatttcttgcttgctcctactttgtgttaccagttacgctatccccgcactgcatttattcgaaagggttgggtgttacggcaactgatcaagctaataatatttacagggttaatgggattcatcattgaacaatatatcaatccgattgtacaaaactctcagcatccattgaacggcgacattttatacgcaattgaacgtgttttaaagctttcagttccaaatttatacgtttggctctgtatgttctactgtttttttcacctttggttgaatatacttgctgagcttcttcgttttggggatcgtgagttttataaagattggtggaatgcacaaactattgaagagtattggagactatggaatatgcctgttcataaatggattgtacggcatctctacttcccatgcttgcgtaatgggatacctaagggtgctgccatcttggttgcgtttttcatgtctgctgtgttccatgagctttgtattgctgttccctgccacattttcaaattttgggcttttattgggatcatgtttcaggtgcccttggtcttactcacgaattacttgcagaacaagttccaaaactcaatggttggaaatataatcttctggtgcttctttagcatccttggtcaacccatgtgtgtattactctactatcatgatgtcatgaatcaaaaggtgaatagcaaataa

SEQ ID NO: 73 - protein, 517 aa, Helianthus annuus
MALLDTSDIGDSTAIRGEIRRRRSVKPDAGFGIGDGLYDSSSSSRTNSSEEEGESLTNGFDENERIRAGDETQTTQENKQKTDQRRDKTSLLQYAYRASSPAHRRIKESPLSSDAIFKQSHAGLFNLCIVVLVAVNGRLIIENLMKYGLLINSNFWFSSRSLRDWPLLMCCVSLLFFPLAAYIVEKLAWKKRISDPVVITLHVIVTTTAILYPVFMILRVDSVVLSGVSLMLCACINWLKLTSFVHTSYDMRSLVNSTDKGETESESLDIELFYDADFKSLVYFLLAPTLCYQLRYPRTAFIRKGWVLRQLIKLIIFTGLMGFIIEQYINPIVQNSQHPLNGDILYAIERVLKLSVPNLYVWLCMFYCFFHLWLNILAELLRFGDREFYKDWWNAQTIEEYWRLWNMPVHKWIVRHLYFPCLRNGIPKGAAILVAFFMSAVFHELCIAVPCHIFKFWAFIGIMFQVPLVLLTNYLQNKFQNSMVGNIIFWCFFSILGQPMCVLLYYHDVMNQKVNSK

SEQ ID NO: 74 - DNA, 1416 bp, Hordeum vulgare
atggccccgcccccgtccgtggccgctgcccacgactgcgacgacccctccctccgcctccgccgcgccgacggcggctcctccggcgtccacggagaggcgcgtccgcaggagcagccgcagcggcagcacgagatgccctgctaccgggcgtccgcgcccgcacaccgccgggtgaaggagagcccgctcagctccgacgccatcttccgacagagccatgcaggtcttctgaatctatgcattgttgtgctgattgcagtgaacagcaggctcattatcgagaacttaatgaagtatggcttattaataagagctggattttggtttagtgcaagatcgctgggagattggccacttctgatgtgctgcctaactttaccaattttcccacttgctgcactcatgaccgagaagtgggctcaaagaaagctcattcgtgatcatgtgtctattcttctccatatcattattacagccactgtccttatctatccggttgttgtgattcttaagtgtgaatcagcagtattatctggatttgtgttaatgttcattgcaagcattacttggttgaagcttgtctcttttgctcatacaaatcatgatataagggtattgtcccaaagtattgaaaagggtgctacacatggcagttccatcgatgaagaaaccattaaaggtccaactaccaacagtgttgtgtatttcatgttggccccaacactttgttaccagccaagttatccccggacagcatttgttaggaaaggctgggtggcccagcagcttataaaatgcatagtttttacaggcttgatgggcttcataattgagcaatacattaatccaattgtgcagaattccaagcatccattgaaaggaaatttcttggatgctattgagagagtcctgaaactctcagtgccgacattgtatgtatggctttgtatgttctattgctttttccatctgtggttgaatattcttgccgaactcctccgttttggtgatcgtgaattctataaggactggtggaatgccagaacagttgaagagtactggagaatgtggaatatgcctgttcataagtggatcgttcgacatatatattttccatgcataaggaatggcttgtcaaagggttgtgccattctcatctcatttcttgtttcagctgtatttcatgagctatgtattgctgttccgtgccacattttcaaactatgggcattttctggaatcatgtttcagattcccctgctattcttgacgaagtatcttcaagataagttcaagaatacaatggcgggcaacatgatattttggttcttcttcagcatagttgggcagccaatgtgtgttctcttgtactaccacgatgtcatgaacagacaagctcagacaaatggctag

SEQ ID NO: 75 - protein, 471 aa, Hordeum vulgare
MAPPPSVAAAHDCDDPSLRLRRADGGSSGVHGEARPQEQPQRQHEMPCYRASAPAHRRVKESPLSSDAIFRQSHAGLLNLCIVVLIAVNSRLIIENLMKYGLLIRAGFWFSARSLGDWPLLMCCLTLPIFPLAALMTEKWAQRKLIRDHVSILLHIIITATVLIYPVVVILKCESAVLSGFVLMFIASITWLKLVSFAHTNHDIRVLSQSIEKGATHGSSIDEETIKGPTTNSVVYFMLAPTLCYQPSYPRTAFVRKGWVAQQLIKCIVFTGLMGFIIEQYINPIVQNSKHPLKGNFLDAIERVLKLSVPTLYVWLCMFYCFFHLWLNILAELLRFGDREFYKDWWNARTVEEYWRMWNMPVHKWIVRHIYFPCIRNGLSKGCAILISFLVSAVFHELCIAVPCHIFKLWAFSGIMFQIPLLFLTKYLQDKFKNTMAGNMIFWFFFSIVGQPMCVLLYYHDVMNRQAQTNG

SEQ ID NO: 76 - DNA, 1434 bp, Oryza sativa
atggccccgcccccctcgctcgcccccgatcgcggcggcggcgaacccgacgacgccctccgcctgcgggcccgcgccgccgccgccgccggtgacgctcccgctccgcagcagcagcaggagcagaggcatcaggagcagcagcagcagctgctctggtaccgcgcgtcggcgcccgcccaccgccgcgtcagggagagccccctcagctccgacgccatcttccgccagagccatgcaggccttctgaacctatgcattgttgtgctggttgctgtgaacagcagacttattattgagaatttaatgaagtatggcctactaattagagctggattttggtttagtggaacatcgctggcagattggcctcttctcatgtgctgtctcactttaccaactttcccgcttgctgcacttatggttgagaagttggctcaaagaaaacttattagtaaacatgtggttattcttctccatatcgttattacaacatctgtccttgtctatccagttgttgtgattctaaagtgtgattcggcagtattatctggatttgtgttgatgtttcttgcaagcattatttggttgaagcttgtttcttttgctcatacaaattatgatataagaatgctgtccaaaagtattgaaaagggcgtgacacatgacatttctatagatccggagaacattaaatggccaacctttaaaaggctatcctacttcatgttggccccaacactttgttaccagccaagttatccccgaactacatatattagaaaaggttgggtggtccgacaactgataaaatgccttgtttttacaggcttgatgggttttataattgagcaatacataaatccaattgtgaagaattcgaagcatccattgaaagggaatttcttgaatgctatagagagagtattgaaattatcagtgccaacattatatgtctggctttgcatgttctactgttttttccatctctggttgaatattcttgctgagctcctctgttttggtgatcgtgaattctacaaggactggtggaatgccaaaacagttgaagagtattggagaatgtggaatatgcctgttcacaagtgggtcattcgacatatatattttccatgcataaggaatggtttttcaaagggtgttgctatcctaatctcgttcctggtttcagctgcatttcatgagctatgtgttgctgttccatgccacatttttaaattctgggcatttattgggatcatgtttcagattcccctggtattcttgacgaaataccttcaagataaattcaataacacaatggtgggcaacatgatattttggttcttcttcagcatcctggggcaaccaatgtgtgttctcttatactaccatgatgtcatgaacaggcaacaagcccaaacaaatagatag

SEQ ID NO: 77 - protein, 538 aa, Oryza sativa
MVGSDGDGDGGGGEAHAPAAPAHHHRRPPRPRGGSGAIVEGFAAALRRRIRSGAAAAARASFGGDSGDEAASGEPSSSSSSSPSRRRGGDSNGAEASSAAGGGGGRGGGGDFSAFTFRAAAPVHRKAKESPLSSDAIFKQSHAGLFNLCIVVLVAVNSRLIIENLMKYGLLIRAGFWFNDKSLRDWPLLMCCLSLPAFPLGAFAVEKLAFNNVITDAVATCLHIFLSTTEIVYPVLVILKCDSAVLSGFLLIFIACIVWLKLVSFAHTNHDIRQLTMGGKKVDNELSTVDMDNLQPPTLGNLIYFMMAPTLCYQPSYPRTSCVRKGWLIRQIILYLIFTGLQGFIIEQYINPIVVNSQHPLKGGLLNAVETVLKLSLPNVYLWLCMFYAFFHLWLSILAEILRFGDREFYKDWWNAKTIDEYWRKWNMPVHKWVVRHIYFPCMRNGISKEVAVLISFLVSAVLHEICVAVPCRILKFWAFLGIMLQIPLIVLTAYLKSKFRDTMVGNMIFWFFFCIYGQPMCLLLYYHDVMNRIEKAR

SEQ ID NO: 78 - DNA, 1473 bp, Sorghum bicolor
atggccccgcccccctccatggccgccgcctccgatcgcgccgtccccggcgccgacgcgaccgaggcgtcctccctccgcctccgccgcgccccctcagccgacgccggcgaccttgccgacgattcctcaggagaccggcgggagaacggcgagccgcaaccgccgcaggagcagcagcagcagcacgagatgctgtactaccgcgcgtcggcgcccgcccaccgccgcgtcaaggagagccccctcagctccgacgccatcttccggcagagccatgctggtcttctgaatctatgcatcgttgttctgattgcagtgaacagcagactcattattgagaatttaatgaagtatggcctattgataagagctggattttggtttagtgcaagatcgctgggtgactggccccttctaatgtgctgcctcactttaccagttttcccacttgttgccctcatggctgagaagctgattagaagaaagctcattggtgaacatgtggttattctactccatatcattattacaacatctgtcattgtctatccagttgttgtgactcttaagtgcgactcagcagtgctatctggattcttgctaatgtttcttgcgagcatcatgtggatgaagcttgtctcttatgcacatacaaattatgatataagggcattgtccaaaagtactgaaaagggtgctgcatatggaaattatgtcgatcctgagagtatgaaagatccaacctttaaaagtctagtgtacttcatgttggccccaacactttgttaccagccaacttatccccgaactacatgtattaggaagggttgggtgacccgacaacttataaagtgcctggtttttacaggcttgatgggcttcataattgagcaatatataaacccaattgtgaagaattccaaacatccactgaaagggaatttcttgaatgctatagaaagagtcttaaaactctcagtgccaacattatatgtatggctttgcatgttctattgcttttttcatttatggctgaacattctagctgaactcctctgtttcggtgaccgtgaattctacaaggactggtggaatgccaaaactgttgaagagtactggaggatgtggaacatgcctgttcataaatggatcatcagacacatatattttccatgtataaggaaaggcttttccaggggtgtagctattctagtctcgtttctggtttcagctgtatttcatgagatatgtattgcggtgccgtgccacattttcaaattctgggcattttctgggatcatgtttcagataccgttggtattcttgacaagatatctccaggctacgttcaagaatataatggtgggcaacatgatattttggttcttcttcagtatagtcgggcagccgatgtgtgtccttttatactaccatgatgtcatgaacaggcaggcccaggcaagtagataa

SEQ ID NO: 79 - protein, 515 aa, Sorghum bicolor
MADTDDAPPAPAVHRRPPRPARGAAAAQGFAAKLRRRLSSGAAAAARASFAADSGDESGPGEPSSSRRRDNGGDASSAADGGRGGAGDFSAFTFRAAAPVHRKAKESPLSSDAIFKQSHAGLFNLCIVVLVAVNSRLIIENLMKYGLLIRSGFWFNATSLRDWPLLMCCLSLPVFPLGAFAVEKLAFNNLITDAAATCFHIFLTTLEIVYPVLVILKCDSAVLSGFVLMFIACIVWLKLVSFAHTNHDIRKLITSGKKVDNELTVADIDNLQAPTLGSLTYFMMAPTLCYQPSYPRTPYVRKGWLVRQVILYLIFTGLQGFIIEQYINPIVVNSQHPLKGGLLNAVETVLKLSLPNVYLWLCMFYCLFHLWLNILAEILRFGDREFYKDWWNAKTIDEYWRKWNMPVHKWMLRHIYFPCIRNGISKEVAAFIAFFVSAVFHELCVAVPCHILKFWAFLGIMLQIPLIILTSYLKNKFNDTMVGNMIFWFFFCIYGQPMCVLLYYHDVMNRTEKTK

SEQ ID NO: 80 - DNA, 1536 bp, Triticum aestivum
atgtcaaaagggaacccagacccgccacctccccggcagcttcctcccttcccacggcgggccgcccaccgaaacccaaaaccccgccccgaaccttccggaacctcccctccagttccacccatggccccgcccccgtccgtggccgctgcccacgatcgcgacgacccctccctccgcctccgccgcgcccctgccgccgacggcgtccacggagaggcggagccgcaggagcagccgcagcggcagcacgagatgccctgctaccgggcgtcggcgcccgcccaccgccgggtcaaggagagcccgcttagctccgacgccatcttccgacagagccatgcaggtcttctgaatctatgcattgttgtgctgattgcagtgaacagcaggctcattatcgagaacttaatgaagtatggcctattaataagagctgggttttggtttagtgcaagatcgctgggagattggccacttctgatgtgctgcctcactttacccattttcccacttgctgcactcatgaccgagaagtgggctcaaagaaagctcatccgtgatcatgtgtctattcttctccatataattattacaaccactgtccttatctatccggttgttgtgattcttaagtgtgaatcagcagtattatctggatttgtgttaatgttcattgcaagcattacttggttgaagcttgtctcttttgctcatacaaattatgatataagggtgttgtcccaaagtattgaaaagggtgctacacatggcagttctatcgatgaggaaaacattaaaggcccaactatcaacagtgttgtgtatttcatgttggccccaacactttgttaccagccaagttatccccggacagcatttactaggaaaggctgggtcactcggcagcttataaaatgcgtagtttttacaggcttgatgggcttcataattgagcaatacattaatccaattgtgcagaattccaagcatccattaaaaggaaatttcttggatgctattgagagagtcttgaaactgtcagtgccaacattatatgtatggctttgtatgttctattcctttttccatctgtggttgaatattcttgccgaactcctccgttttggtgatcgtgaattctacaaggactggtggaatgccaaaacagttgaagagtactggagaatgtggaatatgcctgttcataagtggatcgttcgccatatatattttccatgcataaggaatggcttgtcaaagggttgtgccattctcatcgcatttctggtttcagctgtatttcatgagctatgtattgctgttccatgccacattttcaaattatgggcgttttctggaatcatgtttcagattcccctgctattcttgacgaaatatcttcaagaaaagttcaagaacacaatggtgggcaacatgatattttggttcttcttcagcatagttgggcagccaatgtgtgttctcttgtactaccatgatgtcatgaacagacaggctcagacaaatggctag

SEQ ID NO: 81 - protein, 511 aa, Triticum aestivum
MSKGNPDPPPPRQLPPFPRRAAHRNPKPRPEPSGTSPPVPPMAPPPSVAAAHDRDDPSLRLRRAPAADGVHGEAEPQEQPQRQHEMPCYRASAPAHRRVKESPLSSDAIFRQSHAGLLNLCIVVLIAVNSRLIIENLMKYGLLIRAGFWFSARSLGDWPLLMCCLTLPIFPLAALMTEKWAQRKLIRDHVSILLHIIITTTVLIYPVVVILKCESAVLSGFVLMFIASITWLKLVSFAHTNYDIRVLSQSIEKGATHGSSIDEENIKGPTINSVVYFMLAPTLCYQPSYPRTAFTRKGWVTRQLIKCVVFTGLMGFIIEQYINPIVQNSKHPLKGNFLDAIERVLKLSVPTLYVWLCMFYSFFHLWLNILAELLRFGDREFYKDWWNAKTVEEYWRMWNMPVHKWIVRHIYFPCIRNGLSKGCAILIAFLVSAVFHELCIAVPCHIFKLWAFSGIMFQIPLLFLTKYLQEKFKNTMVGNMIFWFFFSIVGQPMCVLLYYHDVMNRQAQTNG

SEQ ID NO: 82 - DNA, 1485 bp, Zea mays
atggccccgcccccctccatgcctgccgcctccgatcgcgccggccctggccgcgacgcgggcgactcgtcctcccttcgcctccgccgcgccccctcagccgacgccggcgaccttgccggcgattcctcgggaggcttgcgggagaacggcgagccgcaatcgccgacgaatccgccgccgcaggagcagcagcagcacgagatgctatactaccgcgcgtcggcgcccgcccaccgccgcgtcaaggagagccccctcagctctgacgccatcttccggcagagccatgctggtcttctgaatctatgcattgttgttctgatcgcagtgaacagcagactcattattgagaatttaatgaagtatggcctgttgataagagctggattttggtttagtgcaagatcgctgggtgactggccccttctaatgtgctgcctcactctaccagttttcccactagttgcactcatggctgagaagctgatcacaagaaagctcattggtgaacatgtggttattctactccatatcattattacaacatctgccattgtctatccagttgttgtgactcttaagtgtgactcagcagtactatctggatttgtgctaatgtttcttgcgagcatcatgtggatgaagcttgtctcttatgcacatacaaattatgatataagggtattgtccaaaagtactgaaaagggtgctgcatatggaaattatgtcgatcctgagaatatgaaagatccaacctttaaaagtctagtgtactttatgttggccccaacactttgttaccagccaacttatcctcaaactacatgtattagaaagggttgggtgacccagcaactcataaagtgcgtggtttttacaggcttgatgggcttcataattgagcaatatataaacccaattgtgaagaattccaaacatccactgaaagggaattttttgaatgctatagaaagagtcttaaaactctcagtgccaacattatatgtatggctttgcatgttctattgcttttttcatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtatttcatgagatatgtattgcggtgccttgccacattttcaaattctgggcattttctgggatcatgtttcagatacccttggtattcttgacaagatatctccatgctacgttcaagcatgtaatggtgggcaacatgatattttggttcttcttcagtatagtcggacagccgatgtgtgtccttctatactaccatgacgtcatgaacaggcaggcccaggcaagtagatag

SEQ ID NO: 83 - protein, 494 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASR

SEQ ID NO: 84 - DNA, 1539 bp, Elaeis guineensis
atggcggtctcgaagaatccagaaaccctagcccccgatcaagaaccgtcgaaggagtcggatctccgccggaggccggcttcctccccctcctccaccgccgcttcgccggcggttccggactcgtcttcaagaacgagctcttccatcaccgggagctggaccacggcgttagatggagattctggtgcaggagctgtccgcattggtgatccaaaggatcggataggcgaggcgaacgatattggtgaaaagaagaaggcctgttcgggtgaagttccggtggggttcgtggaccggccttcagctccggtgcatgtgagagtggtggagagcccgctgagctcggatacgatctttcaacagagccatgcaggtctcttgaacctttgtgtagtagttctgattgcagttaacagcaggcttattattgagaacttgatgaagtatggtttactaataggaagtggatttttcttcagttcaagattactgagggattggccactacttatatgtagtctcactctacctgttttccctcttggatcttacatggttgaaaagctggcatataagaagttcatttctgaacctgttgttgtctcacttcatgtaatactcataatagctaccatcatgtatccagttttcgtgattctaaggtgcgattctcctattttatctggcatcaatctaatgctctttgtgagctctatttgcctaaagcttgtttcatatgcacatgcaaattacgatttaaggtcatcatccaactctattgataaggggatacacaagtctcaaggtgttagcttcaaaagtttggtgtattttataatggctcccacgctatgttaccagccaagttatcctcggactacgtgcatcagaaagggttgggtgatttgtcagcttgttaagttggtgatatttactggggtgatgggcttcatcattgagcagtacattgacccgattatcaagaattctcaacatcctctaaaaggaaatgtcttaaatgctatggagagagtcttgaagctatcaataccaactttgtacgtgtggctttgtgtattctattgcaccttccatttgtggttaaacattcttgctgagctcctttgttttggtgatcgtgaattctataaagattggtggaatgcaaaaacaattgaagagtattggagaatgtggaacatgcctgttcataaatggatgcttcgccatgtttatcttccatgcatacggaatggtataccaaagggagttgcgatggtcatctcatttttcatttctgctatattccatgagctatgcattggtatcccctgccatatattcaagttctgggctttcatagggataatgtttcaggttccacttgtcatcttgacaaagtacctccagaataaatttaaaagtgccatggtgggaaacatgatcttctggttcttcttcagcatatatggacagcctatgtgtgttctgctttactaccatgatgtgatgaacagaaaagtgggaacagagtaa

SEQ ID NO: 85 - protein, 512 aa, Elaeis guineensis
MAVSKNPETLAPDQEPSKESDLRRRPASSPSSTAASPAVPDSSSRTSSSITGSWTTALDGDSGAGAVRIGDPKDRIGEANDIGEKKKACSGEVPVGFVDRPSAPVHVRVVESPLSSDTIFQQSHAGLLNLCVVVLIAVNSRLIIENLMKYGLLIGSGFFFSSRLLRDWPLLICSLTLPVFPLGSYMVEKLAYKKFISEPVVVSLHVILIIATIMYPVFVILRCDSPILSGINLMLFVSSICLKLVSYAHANYDLRSSSNSIDKGIHKSQGVSFKSLVYFIMAPTLCYQPSYPRTTCIRKGWVICQLVKLVIFTGVMGFIIEQYIDPIIKNSQHPLKGNVLNAMERVLKLSIPTLYVWLCVFYCTFHLWLNILAELLCFGDREFYKDWWNAKTIEEYWRMWNMPVHKWMLRHVYLPCIRNGIPKGVAMVISFFISAIFHELCIGIPCHIFKFWAFIGIMFQVPLVILTKYLQNKFKSAMVGNMIFWFFFSIYGQPMCVLLYYHDVMNRKVGTE

(19) United States
(12) Patent Application Publication
(10) Pub. No.: US 2024/0228985 A1
(43) Pub. Date: Jul. 11, 2024

(54) MODIFIED SEED OIL CONTENT IN PLANTS

(71) Applicant: PIONEER HI-BRED INTERNATIONAL, INC, JOHNSTON, IA (US)

(72) Inventors: ERICKA BERMUDEZ, APTOS, CA (US); KAYLA S. FLYCKT, ANKENY, IA (US); JUSTIN D. JANTES, DES MOINES, IA (US); ZHAN-BIN LIU, CLIVE, IA (US); KEITH R. ROESLER, URBANDALE, IA (US); LAURA L. WAYNE, DES MOINES, IA (US)

(73) Assignee: PIONEER HI-BRED INTERNATIONAL, INC, JOHNSTON, IA (US)

(21) Appl. No.: 18/561,543

(22) PCT Filed: May 16, 2022
(86) PCT No.: PCT/US22/72346

§ 371 Date: Nov. 16, 2023

Related U.S. Application Data
(60) Provisional application No. 63/189,304, filed on May 17, 2021.

(51) Int. Cl. C12N 9/10; C12N 15/82

(52) U.S. Cl. CPC C12N 9/1029; C12N 15/8247; C12N 2501/72; C12N 2510/00; C12Y 203/0102

(57) ABSTRACT

Provided are compositions comprising polynucleotides encoding modified DGAT1 polypeptides. Also provided are recombinant DNA constructs, plants, plant cells, seed, and grain comprising the polynucleotides. Additionally, methods using the polynucleotides in plants to increase seed oil content are also provided herein.

### REFERENCE TO SEQUENCE LISTING SUBMITTED ELECTRONICALLY

The official copy of the sequence listing is submitted electronically via EFS-Web as an ASCII formatted sequence listing with a file named 8544_SequenceListing_ST25 created on Apr. 29, 2021 and having a size of 234 kilobytes and is filed concurrently with the specification. The sequence listing comprised in this ASCII formatted document is part of the specification and is herein incorporated by reference in its entirety.

### FIELD

This disclosure relates to the field of molecular biology.

### BACKGROUND

Plant oils are a major product of oil seed crops such as soybean, sunflower and canola. Oils, such as soybean oil, produced in the US are extracted from seeds and have a major use in food products such as cooking oils, shortenings and margarines. The oils can be refined, bleached and deodorized (RBD) and may be hydrogenated to facilitate use in shortenings.

Accordingly, there is a need to develop compositions and methods to increase oil (e.g., seed oil) content. This disclosure provides such compositions and methods.

### SUMMARY

Provided are modified polynucleotides encoding modified diacylglycerol acyltransferase-1 (DGAT1) polypeptides having at least 60% sequence identity to SEQ ID NO: 2, 50, 67, 69, 71, 73, 75, 77, 79, 81, 83 or 85 and comprising at least one modification selected from the group consisting of a non-histidine at the position corresponding to position 16 of SEQ ID NO: 2, a non-lysine at the position corresponding to position 45 of SEQ ID NO: 2, a non-phenylalanine at the position corresponding to position 75 of SEQ ID NO: 2, a non-phenylalanine at the position corresponding to position 106 of SEQ ID NO: 2, and a non-arginine at the position corresponding to position 200 of SEQ ID NO: 2, wherein when expressed in a plant cell, the polynucleotide increases the fatty acid content of the plant cell compared to a control plant cell (e.g., a plant cell comprising a comparable polynucleotide without the modification). In some embodiments, the encoded polypeptide further comprises at least one modification selected from the group consisting of a non-serine at the position corresponding to position 140 of SEQ ID NO: 2, a non-isoleucine at the position corresponding to position 164 of SEQ ID NO: 2, a non-threonine at the position corresponding to position 210 of SEQ ID NO: 2, a non-tyrosine at the position corresponding to position 225 of SEQ ID NO: 2, a non-aspartic acid at the position corresponding to position 252 of SEQ ID NO: 2, a non-serine at the position corresponding to position 258 of SEQ ID NO: 2, a non-valine at the position corresponding to position 267 of SEQ ID NO: 2, a non-valine at the position corresponding to position 293 of SEQ ID NO: 2, a non-isoleucine at the position corresponding to position 296 of SEQ ID NO: 2, a non-leucine at the position corresponding to position 358 of SEQ ID NO: 2, a non-alanine at the position corresponding to position 416 of SEQ ID NO: 2, a non-isoleucine at the position corresponding to position 473 of SEQ ID NO: 2 and a non-leucine at the position corresponding to position 477 of SEQ ID NO: 2. In some embodiments, the modification is H16L, K45E, F75L, F106S, F106N, F106K, F106D, F106L, F106A, F106W, F106 deletion, S140A, I164M, R200K, T210V, Y225F, D252E, S258T, V267L, V293L, I296V, L358V, A416S, I473S, and L477V.

Also provided are recombinant DNA constructs comprising at least one of the modified polynucleotides encoding a modified DGAT1 polypeptide described herein, optionally operably linked to a heterologous regulatory element (e.g., heterologous promoter).

Further provided are plant cells (e.g., soybean cells) comprising at least one of the modified polynucleotides encoding a modified DGAT1 polypeptide described herein, or a recombinant DNA construct described herein.

Also provided are plants and seeds comprising a plant cell described herein. In some embodiments, the seeds or seeds produced by the plants have an increased fatty acid content as compared to a control seed (e.g., a seed comprising a comparable DGAT1 polynucleotide without the modification).

Also provided are polypeptides encoded by any of the modified polynucleotides described herein.

Provided are methods for producing a plant cell having increased oil content comprising expressing in the plant cell at least one of the modified polynucleotides described herein. In some embodiments, the method comprises expressing in the plant cell a recombinant DNA construct described herein. In some embodiments, the method comprises introducing into the plant cell a targeted genetic modification at an endogenous DGAT gene to produce any of the modified polynucleotides described herein. In certain embodiments, the method further comprises generating a plant from the plant cell, wherein the plant produces seed having increased oil content compared to a control seed not comprising the modified polynucleotide.

Also provided are methods for producing a soybean seed comprising crossing a first soybean line comprising a modified polynucleotide described herein with a second soybean line not comprising the polynucleotide and harvesting the seed produced thereby.

Further provided are polynucleotide modification templates for introducing the targeted genetic modifications described herein.

Also provided is a method of screening for the presence or absence of a modified polynucleotide described herein in a plurality of genomic soybean DNA samples comprising contacting a plurality of genomic soybean DNA samples, at least some of which comprise the polynucleotide, with a first DNA primer molecule and a second DNA primer molecule, performing the nucleic acid amplification, thereby producing a DNA amplicon molecule indicating the presence of the polynucleotide or a wild-type DGAT1 nucleotide sequence, and detecting the DNA amplicon molecules, wherein the presence, absence or size of the DNA amplicon molecule indicates the presence or absence of the polynucleotide in the at least one of the plurality of genomic soybean DNA samples.

### BRIEF DESCRIPTION OF THE DRAWINGS AND THE SEQUENCE LISTING

The disclosure can be more fully understood from the following detailed description and the accompanying drawings and Sequence Listing, which form a part of this application.

FIG. 1 provides a graph showing the experimental results of the Nile Red Lipid staining assay of the top 29 constructs screened in a re-transformation test from the C2500 library. Values are based on Nile Red Fluorescence divided by the OD₆₀₀ of the yeast culture.

FIG. 2 provides a graph of experimental results comparing the oil content in the leaf of N. benthamiana expressing empty vector (EV); GmDGAT1a WT (SEQ ID NO: 2); GmDGAT1a K45E, Y225F, D252, V267L, V293L, I296V, L358V, L477V (SEQ ID NO: 10); GmDGAT1a H16L, S140A, I164M, R200K, L477V (SEQ ID NO: 13); GmDGAT1a S140A, L477V (SEQ ID NO: 28); or GmDGAT1a F75L, F106S, A416S, L477V (SEQ ID NO: 7). Letters indicate statistical significance based on student's t-test, p≤0.05.

FIG. 3 provides a graph of experimental results comparing the oil content in the leaf of N. benthamiana expressing empty vector (EV); GmDGAT1a WT (SEQ ID NO: 2); GmDGAT1a F75L, F106S, A416S, L477V (SEQ ID NO: 7); GmDGAT1a F106S, A416S, L477V (SEQ ID NO: 16); GmDGAT1a F106S (SEQ ID NO: 25); GmDGAT1a F106S, A416S (SEQ ID NO: 30); GmDGAT1a F106S L477V (SEQ ID NO: 19); GmDGAT1a F75L, A416S, L477V (SEQ ID NO: 32); GmDGAT1a A416S, L477V (SEQ ID NO: 34); or GmDGAT1a L477V (SEQ ID NO: 22). Letters indicate statistical significance based on student's t-test, p≤0.05.

FIG. 4 provides a graph of experimental results comparing the oil content in the leaf of N. benthamiana expressing empty vector (EV); GmDGAT1a WT (SEQ ID NO: 2); GmDGAT1a F106S (SEQ ID NO: 25); GmDGAT1a F106N (SEQ ID NO: 36); GmDGAT1a F106K (SEQ ID NO: 38); GmDGAT1a F106D (SEQ ID NO: 40); GmDGAT1a F106L (SEQ ID NO: 42); GmDGAT1a F106A (SEQ ID NO: 44); GmDGAT1a F106W (SEQ ID NO: 46); or GmDGAT1a F106 Deletion (SEQ ID NO: 48). Letters indicate statistical significance based on student's t-test, p<0.05.

FIG. 5 provides a graph of experimental results comparing the oil content in the leaf of N. benthamiana expressing empty vector (EV); GmDGAT1a WT (SEQ ID NO: 2); GmDGAT1a F106S (SEQ ID NO: 25); GmDGAT1a F106S L477V (SEQ ID NO: 19); GmDGAT1b WT (SEQ ID NO: 50); GmDGAT1b F112S (SEQ ID NO: 52); or GmDGAT1b F112S L483V (SEQ ID NO: 54). Letters indicate statistical significance based on student's t-test, p<0.05.

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1
Sequence Listing Description

| Polynucleotide | Polypeptide | | |
|---|---|---|---|
| SEQ ID NO: | SEQ ID NO: | Sequence Name | Modification |
| 1 | 2 | GmDGAT1a WT | |
| 3 | 4 | GmDGAT1a Variant | T210V, I473S, D252E, S258T |
| 5, 6 | 7 | GmDGAT1a Variant | F75L, F106S, A416S, L477V |
| 8, 9 | 10 | GmDGAT1a Variant | K45E, Y225F, D252E, V267L, |
| | | | V293L, I296V, L358V, L477V |
| 11, 12 | 13 | GmDGAT1a Variant | H16L, S140A, I164M, R200K, |
| | | | L477V |
| 14, 15 | 16 | GmDGAT1a Variant | F106S, A416S, L477V |
| 17, 18 | 19 | GmDGAT1a Variant | F106S, L477V |
| 20, 21 | 22 | GmDGAT1a Variant | L477V |
| 23, 24 | 25 | GmDGAT1a Variant | F106S |
| 26, 27 | 28 | GmDGAT1a Variant | S140A, L477V |
| 29 | 30 | GmDGAT1a Variant | F106S, A416S |
| 31 | 32 | GmDGAT1a Variant | F75L, A416S, L477V |
| 33 | 34 | GmDGAT1a Variant | A416S, L477V |
| 35 | 36 | GmDGAT1a Variant | F106N |
| 37 | 38 | GmDGAT1a Variant | F106K |
| 39 | 40 | GmDGAT1a Variant | F106D |
| 41 | 42 | GmDGAT1a Variant | F106L |
| 43 | 44 | GmDGAT1a Variant | F106A |
| 45 | 46 | GmDGAT1a Variant | F106W |
| 47 | 48 | GmDGAT1a Variant | ΔF106 |
| 49 | 50 | GmDGAT1b WT | |
| 51 | 52 | GmDGAT1b | F112S |
| | | Variant | |
| 53 | 54 | GmDGAT1b | F112S, L483V |
| | | Variant | |
| 55 | | KF-Sc-FP1 | |
| 56 | | KF-Sc-RP2 | |
| 57 | | DG1a-FP1 | |
| | 58 | GmDGAT1a Peptide | |
| 59 | | GM-DGAT-CR6 | |
| | 60 | GmDGAT1a | |
| | | genomic | |
| | 61-65 | GmDGAT1 motifs | |
| 66 | 67 | GmDGAT1c WT | |
| 68 | 69 | BnDGAT1a WT | |
| 70 | 71 | GhDGAT1 WT | |
| 72 | 73 | HaDGAT1 WT | |
| 74 | 75 | HvDGATI WT | |
| 76 | 77 | OsDGAT1 WT | |
| 78 | 79 | SbDGAT1b WT | |
| 80 | 81 | TaDGAT1 WT | |
| 82 | 83 | ZmDGAT1-2 WT | |
| 84 | 85 | EgDGAT1-1 WT | |

### DETAILED DESCRIPTION

Provided herein are modified DGAT1 polynucleotides encoding modified DGAT1 polypeptides having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 and comprising a modification at one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more) amino acid residues.

In certain embodiments, the modified DGAT1 polypeptide comprises a modification at one or more amino acid residues corresponding to SEQ ID NO: 2 selected from the group consisting of H16, K45, F75, F106, S140, I164, R200, T210, Y225, D252, S258, V267, V293, I296, L358 A416, I473, and L477 and any combination thereof. In certain embodiments, the modification at one or more amino acid residues corresponding to SEQ ID NO: 2 is selected from the group consisting of H16L, K45E, F75L, F106S, F106N, F106K, F106D, F106L, F106A, F106W, F106 deletion, S140A, I164M, R200K, T210V, Y225F, D252E, S258T, V267L, V293L, I296V, L358V, A416S, I473S and L477V and any combination thereof.

In certain embodiments, the modified DGAT1 polypeptide comprises a modification at one or more amino acid residues corresponding to SEQ ID NO: 2 selected from the group consisting of H16, K45, F75, F106, and R200 and any combination thereof. In certain embodiments, the modification at one or more amino acid residues corresponding to SEQ ID NO: 2 is selected from the group consisting of H16L, K45E, F75L, F106S, F106N, F106K, F106D, F106L, F106A, F106W, F106 deletion, and R200K, and any combination thereof. In certain embodiments, the modified DGAT1 polypeptide further comprises a modification at one or more amino acid residues corresponding to SEQ ID NO: 2 selected from the group consisting of S140, I164, T210, Y225, D252, S258, V267, V293, I296, L358, A416, I473 and L477 and any combination thereof. In certain embodiments, the modification at one or more amino acid residues corresponding to SEQ ID NO: 2 is selected from the group consisting of S140A, I164M, T210V, Y225F, D252E, S258T, V267L, V293L, I296V, L358V, A416S, I473S and L477V and any combination thereof.

In certain embodiments of the modified DGAT1 polypeptides described herein, the modified polypeptide further comprises a deletion of at least 1 and less than 101 amino acids in the N-terminal region corresponding to the region at positions 1 to 101 of SEQ ID NO: 2 (positions 1-107 of SEQ ID NO: 50). The deletion can represent a sequence encoding a polypeptide of at least or at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amino acids, and less than or less than about 107, 106, 105, 100, 95, 90, 85, 80, 75, 70, 65, 60, 55, 50, 45, 40, 35, 30, 25, 20, 15, 10, 9, 8, 7, 6, 5, 4, 3 or 2 amino acids. The deletion can occur in a sequence encoding a polypeptide corresponding to SEQ ID NO: 2 at positions corresponding to position 1 to position 101 of SEQ ID NO: 2. The modified polynucleotide may encode a polypeptide having at least 70%, 75%, 80%, 85%, 90%, or 95% identity to positions 102 to 498 of SEQ ID NO: 2. In certain embodiments, the modified polynucleotide encoding a polypeptide having at least 70%, 75%, 80%, 85%, 90%, or 95% identity to positions 102 to 498 of SEQ ID NO: 2 comprises at least one modification described herein. The deletion can include a deletion corresponding to the N-terminal region of the coding sequence or encoded polypeptide of at least or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 49, 49, 50, 51, 52, 53, 55, 55, 56, 57, 58, 59, 60, 65, 70, 75, 80, 85, 90, 95, 100, 105, 110, 115, 120, 125, 130, 135, 140, 145, 150, 160, 170, 180, 190, 200, 210, 220, 230, 240, 250, 260, 265, 270, 280, 290, or 300 nucleotides and less than or less than about 300, 290, 280, 270, 260, 250, 240, 230, 220, 210, 200, 190, 180, 170, 160, 150, 145, 140, 135, 130, 125, 120, 115, 110, 95, 90, 85, 80, 75, 70, 65, 60, 55, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 nucleotides, for example the deletion can occur in a location corresponding to from position 1 to position 303 of SEQ ID NO: 1.

In certain embodiments of the modified DGAT1 polynucleotides described herein, the modified DGAT1 polynucleotides encodes modified DGAT1 polypeptides which comprise one or more or all three signature motifs such as APTLCYQ (SEQ ID NO: 61, corresponding to position 268-274 of SEQ ID NO: 2), FGDREFYXDWWNA (SEQ ID NO: 62; corresponding to position 364-376 of SEQ ID NO: 2) and LLYYHD (SEQ ID NO: 63; position 484-489 of SEQ ID NO: 2), where X is any amino acid, such as K or Q. Other amino acid motifs in the polypeptides disclosed herein and polynucleotides encoding them include, for example, GFIIEQYINPIVXNSXHPL (SEQ ID NO: 64; corresponding to position 303-321 of SEQ ID NO: 2) and ESPLSSDXIFXQSHAGLXNLCXVVLXAVNXRLIIENLMKYGXLI (SEQ ID NO: 65; corresponding to position 89-132 of SEQ ID NO: 2), wherein X is any amino acid. The polypeptides may include at least 1, 2, 3, 4 or 5 amino acid motifs disclosed herein, and any combination thereof. Such polynucleotides may encode polypeptides having at least about 50%, 55%, 60%, 70%, 75%, 80%, 85%, 90%, or 95% identity to SEQ ID NO:2 and comprise one or more of the motifs disclosed herein.

Table 2 provides the amino acid residues in SEQ ID NOs: 50, 67, 69, 71, 73, 75, 77, 79, 81, 83 and 85 that correspond to amino acid residues (positions) H16, K45, F75, F106, S140, I164, R200, T210, Y225, D252, S258, V267, V293, I296, L358, A416, I473, and L477 of SEQ ID NO: 2.

TABLE 2
Amino Acid Residues Corresponding to the Site of Modification of SEQ ID NO: 2

| GmDGAT1a | GmDGAT1b | GmDGAT1c | BnDGAT1a | GhDGAT1 | HaDGAT1 |
|---|---|---|---|---|---|
| (SEQ ID NO: 2) | (SEQ ID NO: 50) | (SEQ ID NO: 67) | (SEQ ID NO: 69) | (SEQ ID NO: 71) | (SEQ ID NO: 73) |
| Soy | Soy | Soy | Canola | Cotton | Sunflower |
| H16 | H16 | — | — | — | — |
| K45 | K48 | — | — | — | — |
| F75 | F81 | Y94 | F87 | F86 | Y94 |
| F106 | F112 | F125 | F118 | F117 | F125 |
| S140 | S146 | S159 | S152 | S151 | S159 |
| I164 | I170 | I193 | I186 | I185 | I193 |
| R200 | R206 | R219 | R212 | R211 | R219 |
| T210 | T216 | T229 | T223 | A221 | S229 |
| Y225 | Y231 | Y244 | Y237 | Y236 | F244 |
| D252 | D258 | E271 | E258 | — | E271 |
| S258 | S264 | T277 | S264 | S262 | D277 |
| V267 | V273 | V286 | V272 | A271 | L286 |
| V293 | V299 | V312 | A298 | I297 | I312 |
| I296 | I302 | I315 | V301 | I300 | I315 |
| L358 | L364 | L377 | L363 | L362 | L377 |
| A416 | A422 | A435 | A421 | A420 | A435 |
| I473 | I479 | I492 | A478 | I477 | F492 |
| L477 | L483 | L496 | F481 | L481 | L496 |
| HvDGAT1 | OsDGAT1 | SbDGAT1b | TaDGAT1 | ZmDGAT1-2 | EgDGAT1-1 |
| (SEQ ID NO: 75) | (SEQ ID NO: 77) | (SEQ ID NO: 79) | (SEQ ID NO: 81) | (SEQ ID NO: 83) | (SEQ ID NO: 85) |
| Barley | Rice | Sorghum | Wheat | Maize | Palm |
| — | — | — | — | — | — |
| — | — | — | — | — | — |
| — | L52 | L66 | — | L70 | F97 |
| L78 | L83 | L97 | L118 | L101 | L128 |
| A112 | G118 | A131 | A152 | A135 | S162 |
| I146 | I151 | I165 | I186 | I189 | I196 |
| K172 | K177 | K191 | K212 | K195 | R222 |
| V182 | L250 | V227 | V222 | V205 | N232 |
| F197 | F202 | Y216 | F237 | Y220 | Y247 |
| E224 | D292 | D269 | E264 | E247 | H266 |
| T230 | T298 | T275 | T270 | T253 | S272 |
| L239 | L244 | L258 | L279 | L262 | M281 |
| I265 | I270 | I284 | I305 | I288 | V307 |
| I268 | L273 | L287 | V308 | V291 | V310 |
| L330 | L335 | L349 | L370 | V353 | L372 |
| S388 | S393 | S407 | A428 | S411 | S430 |
| F445 | F513 | F490 | F485 | F468 | F487 |
| V449 | L454 | V468 | V489 | V472 | Y491 |

As used herein, a “mutated polynucleotide”, “modified polynucleotide”, “mutated polypeptide”, or “modified polypeptide” is a polynucleotide or polypeptide that has been altered through human intervention. Such a “mutated polynucleotide”, “modified polynucleotide”, “mutated polypeptide”, or “modified polypeptide” has a sequence that differs from the sequence of the corresponding non-mutated or non-modified polynucleotide or polypeptide by at least one nucleotide or amino acid. In certain embodiments of the disclosure, the mutated polynucleotide or modified polynucleotide comprises an alteration that results from a guide polynucleotide/Cas endonuclease system as disclosed herein. A mutated or modified plant is a plant comprising a mutated or modified polynucleotide or polypeptide.

The “modification” at the indicated residue (position) of the DGAT1 polypeptides provided herein may be independently selected from an amino acid substitution, an amino acid deletion, or an amino acid addition. When the DGAT1 polypeptide comprises two or more modifications, each modification may be the same type of modification (i.e., substitution mutation, deletion mutation, or addition mutation) or they may be a combination of two or more types of modifications (e.g., a deletion mutation at one residue and a substation mutation at another residue).

As used herein an “amino acid deletion,” “deletion mutation,” or the like, refers to a mutation in which the indicated amino acid residue is removed from the polypeptide sequence, so that, when aligned to the reference sequence (e.g., SEQ ID NO: 2) the mutated sequence does not have an amino acid corresponding to the indicated position of the reference sequence. An “amino acid addition,” “addition mutation,” or the like, refers to a mutation in which at least one amino acid residue is added to the polypeptide sequence, so that, when aligned to the reference sequence (e.g., SEQ ID NO: 2) the mutated sequence contains an additional amino acid corresponding to the indicated position of the reference sequence.

An “amino acid substitution,” “substitution mutation,” or the like, refers to a modification in which the indicated amino acid residue is replaced with a different amino acid residue, so that, when aligned to the reference sequence (e.g., SEQ ID NO: 2) the mutated sequence does not have the same amino acid at the indicated position. When the amino acid residue is substituted for a residue that has similar properties (e.g., size, charge, and/or hydrophobicity) the substitution is referred to as a conservative amino substitution. Conservative amino acid substitutions are well known in the art. For example, the following six groups contain amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Alternatively, when the amino acid residue is substituted for an amino acid that has dissimilar properties the modification is referred to as a radical amino acid substitution.

The type of amino acid substitution (i.e., conservative or radical) in the modified DGAT1 polypeptides provided herein is not particularly limited, such that the modified DGAT1 polypeptides provided herein may contain all conservative amino acid substitutions, all radical amino acid substitutions, or a combination of radical and conservative amino acid substitutions.

In certain embodiments, the modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 comprises one or more modifications selected from a non-histidine at the position corresponding to position 16 of SEQ ID NO: 2 (i.e., does not comprise an H at the position corresponding to position 16 of SEQ ID NO: 2), a non-lysine at the position corresponding to position 45 of SEQ ID NO: 2, a non-phenylalanine, a non-leucine, and/or a non-tyrosine at the position corresponding to position 75 of SEQ ID NO: 2, a non-phenylalanine and/or a non-leucine at the position corresponding to position 106 of SEQ ID NO: 2, a non-serine, a non-alanine, and/or a non-glycine at the position corresponding to position 140 of SEQ ID NO: 2, a non-isoleucine at the position corresponding to position 164 of SEQ ID NO: 2, a non-arginine and/or a non-lysine at the position corresponding to position 200 of SEQ ID NO: 2, a non-threonine, a non-alanine, a non-serine, a non-valine, a non-leucine, and/or a non-asparagine at the position corresponding to position 210 of SEQ ID NO: 2, a non-tyrosine and/or a non-phenylalanine at the position corresponding to position 225 of SEQ ID NO: 2, a non-aspartic acid, a non-histidine and/or a non-glutamic acid at the position corresponding to position 252 of SEQ ID NO: 2, a non-serine, a non-aspartic acid, and/or a non-threonine at the position corresponding to position 258 of SEQ ID NO: 2, a non-valine, a non-alanine, a non-leucine and/or a non-methionine at the position corresponding to position 267 of SEQ ID NO: 2, a non-valine, a non-alanine, and/or a non-isoleucine at the position corresponding to position 293 of SEQ ID NO: 2, a non-isoleucine, a non-leucine, and/or a non-valine at the position corresponding to position 296 of SEQ ID NO: 2, a non-leucine and/or a non-valine at the position corresponding to position 358 of SEQ ID NO: 2, a non-alanine and/or a non-serine at the position corresponding to position 416 of SEQ ID NO: 2, a non-isoleucine, a non-alanine, and/or a non-phenylalanine at the position corresponding to position 473 of SEQ ID NO: 2, and a non-leucine and/or a non-valine at the position corresponding to position 477, or any combination thereof.

In certain embodiments, the modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 comprises one or more modifications selected from a non-histidine at the position corresponding to position 16 of SEQ ID NO: 2, a non-lysine at the position corresponding to position 45 of SEQ ID NO: 2, a non-phenylalanine, a non-leucine, and/or a non-tyrosine at the position corresponding to position 75 of SEQ ID NO: 2, a non-phenylalanine and/or a non-leucine at the position corresponding to position 106 of SEQ ID NO: 2, and a non-arginine and/or a non-lysine at the position corresponding to position 200 of SEQ ID NO: 2, or any combination thereof. In certain embodiments, the modified DGAT1 polypeptide further comprises one or more modifications selected from a non-serine, a non-alanine, and/or a non-glycine at the position corresponding to position 140 of SEQ ID NO: 2, a non-isoleucine at the position corresponding to position 164 of SEQ ID NO: 2, a non-threonine, a non-alanine, a non-serine, a non-valine, a non-leucine, and/or a non-asparagine at the position corresponding to position 210 of SEQ ID NO: 2, a non-tyrosine and/or a non-phenylalanine at the position corresponding to position 225 of SEQ ID NO: 2, a non-aspartic acid, a non-histidine and/or a non-glutamic acid at the position corresponding to position 252 of SEQ ID NO: 2, a non-serine, a non-aspartic acid, and/or a non-threonine at the position corresponding to position 258 of SEQ ID NO: 2, a non-valine, a non-alanine, a non-leucine and/or a non-methionine at the position corresponding to position 267 of SEQ ID NO: 2, a non-valine, a non-alanine, and/or a non-isoleucine at the position corresponding to position 293 of SEQ ID NO: 2, a non-isoleucine, a non-leucine, and/or a non-valine at the position corresponding to position 296 of SEQ ID NO: 2, a non-leucine and/or a non-valine at the position corresponding to position 358 of SEQ ID NO: 2, a non-alanine and/or a non-serine at the position corresponding to position 416 of SEQ ID NO: 2, a non-isoleucine, a non-alanine, and/or a non-phenylalanine at the position corresponding to position 473 of SEQ ID NO: 2, and a non-leucine, non-phenylalanine, a non-tyrosine and/or a non-valine at the position corresponding to position 477, or any combination thereof.

In certain embodiments, the modified DGAT1 polypeptides having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 comprises an L at the position corresponding to position 16 of SEQ ID NO: 2, an E at the position corresponding to position 45 of SEQ ID NO: 2, an S, K, D, L, A, or W at the position corresponding to position 106 of SEQ ID NO: 2, an L at the position corresponding to position 75 of SEQ ID NO: 2, and/or a K at the position corresponding to position 200 of SEQ ID NO: 2, or any combination thereof. In certain embodiments, the modified DGAT1 polypeptide further comprises an A at the position corresponding to position 140 of SEQ ID NO: 2, an M at the position corresponding to position 164 of SEQ ID NO: 2, a V at the position corresponding to position 210 of SEQ ID NO: 2, an F at the position corresponding to position 225 of SEQ ID NO: 2, an E at the position corresponding to position 252 of SEQ ID NO: 2, a T at the position corresponding to position 258 of SEQ ID NO: 2, an L at the position corresponding to position 267 of SEQ ID NO: 2, an L at the position corresponding to position 293 of SEQ ID NO: 2, a V at the position corresponding to position 296 of SEQ ID NO: 2, a V at the position corresponding to position 358 of SEQ ID NO: 2, an S at the position corresponding to position 416 of SEQ ID NO: 2, an S at the position corresponding to position 473 of SEQ ID NO: 2, and/or a V at the position corresponding to position 477 of SEQ ID NO: 2, or any combination thereof.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise a T, A, S, V, L and/or N at the position corresponding to position 210 of SEQ ID NO: 2 (e.g., comprises a non-threonine at the position corresponding to position 210 of SEQ ID NO: 2), a D, E and/or H at the position corresponding to position 252 of SEQ ID NO: 2, an S, T and/or D at the position corresponding to position 258 of SEQ ID NO: 2, and a I, A and/or F at the position corresponding to position 473 of SEQ ID NO: 2. In certain embodiments the modified DGAT1 polypeptide comprises a V at the position corresponding to position 210 of SEQ ID NO: 2, an E at the position corresponding to position 252 of SEQ ID NO: 2, a T at the position corresponding to position 258 of SEQ ID NO: 2, and an S at the position corresponding to position 473 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F, L and/or Y at the position corresponding to position 75 of SEQ ID NO: 2, an F and/or L at the position corresponding to position 106 of SEQ ID NO: 2, an A and/or S at the position corresponding to position 416 of SEQ ID NO: 2, and an L, F, V and/or Y at the position corresponding to position 477 of SEQ ID NO: 2. In certain embodiments, the modified DGAT1 polypeptide comprises an L at the position corresponding to position 75 of SEQ ID NO: 2, an S at the position corresponding to position 106 of SEQ ID NO: 2, an S at the position corresponding to position 416 of SEQ ID NO: 2, and a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise a K at the position corresponding to position 45 of SEQ ID NO: 2, a Y and/or F at the position corresponding to position 225 of SEQ ID NO: 2, a D, E and/or H at the position corresponding to position 252 of SEQ ID NO: 2, a V, A, L and/or M at the position corresponding to position 267 of SEQ ID NO: 2, a V, A and/or I at the position corresponding to position 293 of SEQ ID NO: 2, a I, L and/or V at the position corresponding to position 296 of SEQ ID NO: 2, an L and/or V at the position corresponding to position 358 of SEQ ID NO: 2, and an L, F, V and/or Y at the position corresponding to position 477 of SEQ ID NO: 2. In certain embodiments the DGAT1 polypeptide comprises an E at the position corresponding to position 45 of SEQ ID NO: 2, a F at the position corresponding to position 225 of SEQ ID NO: 2, a E at the position corresponding to position 252 of SEQ ID NO: 2, a L at the position corresponding to position 267 of SEQ ID NO: 2, a L at the position corresponding to position 293 of SEQ ID NO: 2, a V at the position corresponding to position 296 of SEQ ID NO: 2, an V at the position corresponding to position 358 of SEQ ID NO: 2, and an V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an H at the position corresponding to position 16 of SEQ ID NO: 2, an S, A and/or G at the position corresponding to position 140 of SEQ ID NO: 2, a I at the position corresponding to position 164 of SEQ ID NO: 2, an R and/or K at the position corresponding to position 200 of SEQ ID NO: 2, and an L, F, V and/or Y at the position corresponding to position 477 of SEQ ID NO: 2. In certain embodiments, the DGAT1 polypeptide comprises an L at the position corresponding to position 16 of SEQ ID NO: 2, an A at the position corresponding to position 140 of SEQ ID NO: 2, an M at the position corresponding to position 164 of SEQ ID NO: 2, a K at the position corresponding to position 200 of SEQ ID NO: 2, and a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F and/or L at the position corresponding to position 106 of SEQ ID NO: 2, an A and/or S at the position corresponding to position 416 of SEQ ID NO: 2, and an L, F, V and/or Y at the position corresponding to position 477 of SEQ ID NO: 2. In certain embodiments, the modified DGAT1 polypeptide comprises an S at the position corresponding to position 106 of SEQ ID NO: 2, an S at the position corresponding to position 416 of SEQ ID NO: 2, and a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F and/or L at the position corresponding to position 106 of SEQ ID NO: 2 and an L, F, V and/or Y at the position corresponding to position 477 of SEQ ID NO: 2. In certain embodiments, the modified DGAT1 polypeptide comprises an S at the position corresponding to position 106 of SEQ ID NO: 2 and a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an L, F, V and/or Y at the position corresponding to position 477 of SEQ ID NO: 2. In certain embodiments, the modified DGAT1 polypeptide comprises a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F and/or L at the position corresponding to position 106 of SEQ ID NO: 2. In certain embodiments, the modified DGAT1 polypeptide comprises a S, N, K, D, L, A, or W at the position corresponding to position 106 of SEQ ID NO: 2. In certain embodiments, the modified DGAT1 polypeptide comprises a deletion at the position corresponding to position 106 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an S, A and/or G at the position corresponding to position 140 of SEQ ID NO: 2 and an L, F, V and/or Y at the position corresponding to position 477 of SEQ ID NO: 2. In certain embodiments, the modified DGAT1 polypeptide comprises an A at the position corresponding to position 140 of SEQ ID NO: 2 and a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F and/or L at the position corresponding to position 106 of SEQ ID NO: 2 and an A and/or S at the position corresponding to position 416 of SEQ ID NO: 2. In certain embodiments, the modified DGAT1 polypeptide comprises an S at the position corresponding to position 106 of SEQ ID NO: 2 and an S at the position corresponding to position 416 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F, L and/or Y at the position corresponding to position 75 of SEQ ID NO: 2, an A and/or S at the position corresponding to position 416 of SEQ ID NO: 2, and an L, F, V and/or Y at the position corresponding to position 477 of SEQ ID NO: 2. In certain embodiments, the modified DGAT1 polypeptide comprises an L at the position corresponding to position 75 of SEQ ID NO: 2, an S at the position corresponding to position 416 of SEQ ID NO: 2, and a V at the position corresponding to position 477 of SEQ ID NO: 2.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an A and/or S at the position corresponding to position 416 of SEQ ID NO: 2 and an L, F, V and/or Y at the position corresponding to position 477 of SEQ ID NO: 2. In certain embodiments, the modified DGAT1 polypeptide comprises an S at the position corresponding to position 416 of SEQ ID NO: 2 and a V at the position corresponding to position 477 of SEQ ID NO: 2.

In certain embodiments, the modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 comprises one or more modifications selected from a non-histidine at the position corresponding to position 16 of SEQ ID NO: 50, a non-lysine at the position corresponding to position 48 of SEQ ID NO: 50, a non-phenylalanine at the position corresponding to position 81 of SEQ ID NO: 50, a non-phenylalanine at the position corresponding to position 112 of SEQ ID NO: 50, a non-serine at the position corresponding to position 146 of SEQ ID NO: 50, a non-isoleucine at the position corresponding to position 170 of SEQ ID NO: 50, a non-arginine at the position corresponding to position 206 of SEQ ID NO: 50, a non-tyrosine at the position corresponding to position 231 of SEQ ID NO: 50, a non-valine at the position corresponding to position 273 of SEQ ID NO: 50, a non-valine at the position corresponding to position 299 of SEQ ID NO: 50, a non-isoleucine at the position corresponding to position 302 of SEQ ID NO: 50, a non-alanine at the position corresponding to position 422 of SEQ ID NO: 50, a non-leucine at the position corresponding to position 483 of SEQ ID NO: 50, a non-threonine at the position corresponding to position 216 of SEQ ID NO: 50, a non-aspartic acid at the position corresponding to position 258 of SEQ ID NO: 50, a non-serine at the position corresponding to position 264 of SEQ ID NO: 50, a non-leucine at the position corresponding to position 364 of SEQ ID NO: 50, and a non-isoleucine at the position corresponding to position 479 of SEQ ID NO: 50, or any combination thereof.

In certain embodiments, the modified DGAT1 polypeptides having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 comprises an L at the position corresponding to position 16 of SEQ ID NO: 50, an E at the position corresponding to position 48 of SEQ ID NO: 50, an S, K, D, L, A, or W at the position corresponding to position 112 of SEQ ID NO: 50, an L at the position corresponding to position 81 of SEQ ID NO: 50, an A at the position corresponding to position 146 of SEQ ID NO: 50, an M at the position corresponding to position 170 of SEQ ID NO: 50, a K at the position corresponding to position 206 of SEQ ID NO: 50, an F at the position corresponding to position 231 of SEQ ID NO: 50, an L at the position corresponding to position 273 of SEQ ID NO: 50, an L at the position corresponding to position 299 of SEQ ID NO: 50, a V at the position corresponding to position 302 of SEQ ID NO: 50, an S at the position corresponding to position 422 of SEQ ID NO: 50, a V at the position corresponding to position 483 of SEQ ID NO: 50, a V at the position corresponding to position 216 of SEQ ID NO: 50, an E at the position corresponding to position 258 of SEQ ID NO: 50, a T at the position corresponding to position 264 of SEQ ID NO: 50, a V at the position corresponding to position 364 of SEQ ID NO: 50, or an S at the position corresponding to position 479 of SEQ ID NO: 50 or any combination thereof.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise a T at the position corresponding to position 216 of SEQ ID NO: 50, a D at the position corresponding to position 258 of SEQ ID NO: 50, an S at the position corresponding to position 264 of SEQ ID NO: 50, and a I at the position corresponding to position 479 of SEQ ID NO: 50. In certain embodiments the modified DGAT1 polypeptide comprises a V at the position corresponding to position 216 of SEQ ID NO: 50, an E at the position corresponding to position 258 of SEQ ID NO: 50, a T at the position corresponding to position 264 of SEQ ID NO: 50, and an S at the position corresponding to position 479 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F at the position corresponding to position 81 of SEQ ID NO: 50, an F at the position corresponding to position 112 of SEQ ID NO: 50, an A at the position corresponding to position 422 of SEQ ID NO: 50, and an L at the position corresponding to position 483 of SEQ ID NO: 50. In certain embodiments, the modified DGAT1 polypeptide comprises an L at the position corresponding to position 81 of SEQ ID NO: 50, an S at the position corresponding to position 112 of SEQ ID NO: 50, an S at the position corresponding to position 422 of SEQ ID NO: 50, and a V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise a K at the position corresponding to position 48 of SEQ ID NO: 50, a Y at the position corresponding to position 231 of SEQ ID NO: 50, a D at the position corresponding to position 258 of SEQ ID NO: 50, a V at the position corresponding to position 273 of SEQ ID NO: 50, a V at the position corresponding to position 299 of SEQ ID NO: 50, a I at the position corresponding to position 302 of SEQ ID NO: 50, an L at the position corresponding to position 364 of SEQ ID NO: 50, and an L at the position corresponding to position 483 of SEQ ID NO: 50. In certain embodiments the DGAT1 polypeptides comprises a E at the position corresponding to position 48 of SEQ ID NO: 50, a F at the position corresponding to position 231 of SEQ ID NO: 50, a E at the position corresponding to position 258 of SEQ ID NO: 50, a L at the position corresponding to position 273 of SEQ ID NO: 50, a L at the position corresponding to position 299 of SEQ ID NO: 50, a V at the position corresponding to position 302 of SEQ ID NO: 50, an V at the position corresponding to position 364 of SEQ ID NO: 50, and an V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an H at the position corresponding to position 16 of SEQ ID NO: 50, an S at the position corresponding to position 146 of SEQ ID NO: 50, a I at the position corresponding to position 170 of SEQ ID NO: 50, an R at the position corresponding to position 206 of SEQ ID NO: 50, and an L at the position corresponding to position 483 of SEQ ID NO: 50. In certain embodiments, the DGAT1 polypeptide comprises an L at the position corresponding to position 16 of SEQ ID NO: 50, an A at the position corresponding to position 146 of SEQ ID NO: 50, an M at the position corresponding to position 170 of SEQ ID NO: 50, a K at the position corresponding to position 206 of SEQ ID NO: 50, and an V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F at the position corresponding to position 112 of SEQ ID NO: 50, an A at the position corresponding to position 422 of SEQ ID NO: 50, and an L at the position corresponding to position 483 of SEQ ID NO: 50. In certain embodiments, the modified DGAT1 polypeptide comprises an S at the position corresponding to position 112 of SEQ ID NO: 50, an S at the position corresponding to position 422 of SEQ ID NO: 50, and a V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F at the position corresponding to position 112 of SEQ ID NO: 50 and an L at the position corresponding to position 483 of SEQ ID NO: 50. In certain embodiments, the modified DGAT1 polypeptide comprises an S at the position corresponding to position 112 of SEQ ID NO: 50 and a V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an L at the position corresponding to position 483 of SEQ ID NO: 50. In certain embodiments, the modified DGAT1 polypeptide comprises a V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F at the position corresponding to position 112 of SEQ ID NO: 50. In certain embodiments, the modified DGAT1 polypeptide comprises a S, N, K, D, L, A, or W at the position corresponding to position 112 of SEQ ID NO: 50. In certain embodiments, the modified DGAT1 polypeptide comprises a deletion at the position corresponding to position 112 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an S at the position corresponding to position 146 of SEQ ID NO: 50 and an L at the position corresponding to position 483 of SEQ ID NO: 50. In certain embodiments, the modified DGAT1 polypeptide comprises an A at the position corresponding to position 146 of SEQ ID NO: 50 and a V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F at the position corresponding to position 112 of SEQ ID NO: 50 and an A at the position corresponding to position 422 of SEQ ID NO: 50. In certain embodiments, the modified DGAT1 polypeptide comprises an S at the position corresponding to position 112 of SEQ ID NO: 50 and an S at the position corresponding to position 422 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an F at the position corresponding to position 81 of SEQ ID NO: 50, an A at the position corresponding to position 422 of SEQ ID NO: 50, and an L at the position corresponding to position 483 of SEQ ID NO: 50. In certain embodiments, the modified DGAT1 polypeptide comprises an L at the position corresponding to position 81 of SEQ ID NO: 50, an S at the position corresponding to position 422 of SEQ ID NO: 50, and a V at the position corresponding to position 483 of SEQ ID NO: 50.

In certain embodiments, the encoded modified DGAT1 polypeptide having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2, 50, 67, 69, 71, 73, 75, 77, 79, 81, 83 or 85 does not comprise an A at the position corresponding to position 422 of SEQ ID NO: 50 and an L at the position corresponding to position 483 of SEQ ID NO: 50. In certain embodiments, the modified DGAT1 polypeptide comprises an S at the position corresponding to position 422 of SEQ ID NO: 50 and a V at the position corresponding to position 483 of SEQ ID NO: 50.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions T210, D252, S258, and I473 of SEQ ID NO: 2. In certain embodiments, the amino acid modifications are T210V, D252E, S258T, and I473S.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions F75, F106, A416 and L477 of SEQ ID NO: 2. In certain embodiments, the amino acid modifications are F75L, F106S, A416S and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions K45, Y225, D252, V267, V293, I296, L358, and L477 of SEQ ID NO: 2. In certain embodiments, the amino acid modifications are K45E, Y225F, D252E, V267L, V293L, I296V, L358V, and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions H16, S140, I164, R200, and L477 of SEQ ID NO: 2. In certain embodiments, the amino acid modifications are H16L, S140A, I164M, R200K, and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions F106, A416, and L477 of SEQ ID NO: 2. In certain embodiments, the amino acid modifications are F106S, A416S, and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions F106 and L477 of SEQ ID NO: 2. In certain embodiments, the amino acid modifications are F106S and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to position L477 of SEQ ID NO: 2. In certain embodiments, the amino acid modification is L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to position F106 of SEQ ID NO: 2. In certain embodiments, the amino acid modification is F106S, F106N, F106K, F106D, F106L, F106A, or F106W. In certain embodiments, the amino acid modification is a deletion of F106

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions S140 and L477 of SEQ ID NO: 2. In certain embodiments, the amino acid modifications are S140A and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions F106 and A416 of SEQ ID NO: 2. In certain embodiments, the amino acid modifications are F106S and A416A.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions F75, A416, and L477 of SEQ ID NO: 2. In certain embodiments, the amino acid modifications are F75L, A416S, and L477V.

In certain embodiments, the modified DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises, consists essentially of, or consists of an amino acid modification at the amino acid residue corresponding to positions A416 and L477 of SEQ ID NO: 2. In certain embodiments, the amino acid modifications are A416S and L477V.

As should be understood by those of ordinary skill in the art, a modification of, for example, F106S of SEQ ID NO: 2 indicates a substitution modification in which the phenylalanine (F) at position 106 of SEQ ID NO: 2, or the amino acid in SEQ ID ON: 50 which corresponds to position 106 of SEQ ID NO: 2, is mutated to a serine (S).

In certain embodiments, the modification at one or more amino acid residues is at an amino acid residue corresponding to SEQ ID NO: 50 selected from the group consisting of H16, K48, F81, F112, S146, I170, R206, T216, Y231, D258, S264, V273, V299, I302, L364, A422, I479, and L483 and any combination thereof.

In certain embodiments, the modification at one or more amino acid residues corresponding to SEQ ID NO: 50 is selected from the group consisting of H16L, K48E, F81L, F112S, F112N, F112K, F112D, F112L, F112A, F112W, S146A, I170M, R206K, T216V, Y231F, D258E, S264T, V273L, V299L, I302V, L364V, A422S, I479S, and L483V and any combination thereof.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions T216, D258, S264, and I479 of SEQ ID NO: 50. In certain embodiments, the amino acid modifications are T216V, D258E, S264T, and I479S.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions F81, F112, A422 and L483 of SEQ ID NO: 50. In certain embodiments, the amino acid modifications are F81L, F112S, A422S and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions K48, Y231, D258, V273, V299, I302, L364, and L483 of SEQ ID NO: 50. In certain embodiments, the amino acid modifications are K48E, Y231F, D258E, V273L, V299L, I302V, L364V, and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions H16, S146, I170, R206, and L483 of SEQ ID NO: 50. In certain embodiments, the amino acid modifications are H16L, S146A, I170M, R206K, and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions F112, A422, and L483 of SEQ ID NO: 50. In certain embodiments, the amino acid modifications are F112S, A422S, and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions F112 and L483 of SEQ ID NO: 50. In certain embodiments, the amino acid modifications are F112S and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to position L483 of SEQ ID NO: 50. In certain embodiments, the amino acid modification is L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to position F112 of SEQ ID NO: 50. In certain embodiments, the amino acid modification is F112S, F112N, F112K, F112D, F112L, F112A, or F112W. In certain embodiments, the amino acid modification is a deletion of F112

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions S146 and L483 of SEQ ID NO: 50. In certain embodiments, the amino acid modifications are S146A and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions F112 and A422 of SEQ ID NO: 50. In certain embodiments, the amino acid modifications are F112S and A422S.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions F81, A422, and L483 of SEQ ID NO: 50. In certain embodiments, the amino acid modifications are F81L, A422S, and L483V.

In certain embodiments, the DGAT1 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprises, consists essentially of, or consists of an amino acid modification at the amino acid residue corresponding to positions A422 and L483 of SEQ ID NO: 50. In certain embodiments, the amino acid modifications are A422S and L483V.

In certain embodiments, the modified polynucleotide encodes a DGAT1 polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 4, 7, 10, 13, 16, 19, 22, 25, 28, 30, 32, 34, 36, 38, 40, 42, 44, 46, 48, 52, 54, 67, 69, 71, 73, 75, 77, 79, 81, 83 or 85, wherein the polypeptide comprises at least one of the amino acid modifications described herein.

In certain embodiments the modified DGAT1 polynucleotide comprises a nucleotide sequence having at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1, 3, 5, 6, 8, 9, 11, 12, 14, 15, 17, 18, 20, 21, 23, 24, 26, 27, 29, 31, 33, 35, 37, 39, 41, 43, 45, 47, 49, 51, 53, 60, 66, 68, 70, 72, 74, 76, 78, 80, 82 or 84, wherein the polynucleotide sequence encodes a DGAT1 polypeptide comprising at least one amino acid modification described herein.

In certain embodiments, when the modified DGAT1 polynucleotide encoding the modified DGAT1 polypeptide is expressed in a plant cell the fatty acid (oil) content of the plant cell is increased as compared to a control plant cell (e.g., plant cell comprising a comparable polynucleotide without the modification). The increase in fatty acid content can be any increased level described herein (e.g., at least about a 5% increase)

In certain embodiments, the modified DGAT1 polypeptide further comprises one or more additional modifications that when expressed in a plant cell increase the fatty acid content of the plant cell. For example, DGAT1 modifications described in WO 2019/232182.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

Also provided herein are polynucleotide modification templates for generating the modified polynucleotides described herein (e.g., modification of an endogenous DGAT1 gene). As used herein, “polynucleotide modification template” refers to a polynucleotide sequence that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited. In certain embodiments, the flanking sequences upstream and/or downstream from the at least one nucleotide modification independently comprise at least 5, 10, 25, 50, 100, 150, 250, 500 or 1000 contiguous nucleotides.

In certain embodiments, the polynucleotide modification template comprises a polynucleotide sequence encoding a region of SEQ ID NO: 2, 50, 67, 69, 71, 73, 75, 77, 79, 81, 83 or 85 and comprising a modification at one or more amino acid residues described herein (e.g., H16, K45, F75, F106, S140, I164, R200, T210, Y225, D252, S258, V267, V293, I296, L358, A416, I473, and L477 of SEQ ID NO: 2). In certain embodiments, the polynucleotide modification template comprises a polynucleotide sequence encoding a region of SEQ ID NO: 2, 50, 67, 69, 71, 73, 75, 77, 79, 81, 83 or 85 comprising a modification at one or more amino acid residues selected from the group consisting of H16L, K45E, F75L, F106S, F106N, F106K, F106D, F106L, F106A, F106W, S140A, I164M, R200K, T210V, Y225F, D252E, S258T, V267L, V293L, I296V, L358V, A416S, I473S, and L477V. In certain embodiments, the region of SEQ ID NO: 2, 50, 67, 69, 71, 73, 75, 77, 79, 81, 83 or 85 comprises at least 5, 10, 20, 30, 40, 50, 60, 70, 80, 90, 100, 200, 250, or 350 contiguous amino acids.

Further provided is a recombinant DNA construct comprising any of the modified DGAT1 polynucleotides or polynucleotide modification templates described herein. In certain embodiments, the recombinant DNA construct further comprises at least one regulatory element. In certain embodiments, the at least one regulatory element of the recombinant DNA construct comprises a promoter, preferably a heterologous promoter.

As used herein, a “recombinant DNA construct” comprises two or more operably linked DNA segments which are not found operably linked in nature. Non-limiting examples of recombinant DNA constructs include a polynucleotide of interest operably linked to heterologous sequences, also referred to as “regulatory elements,” which aid in the expression, autologous replication, and/or genomic insertion of the sequence of interest. Such regulatory elements include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The modified DGAT1 polynucleotides described herein can be provided for expression in a plant of interest or an organism of interest. The cassette can include 5′ and 3′ regulatory sequences operably linked to a modified DGAT1 polynucleotide. “Operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the modified DGAT1 polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a modified DGAT1 polynucleotide described herein, and a transcriptional and translational termination region (e.g., termination region) functional in plants. The regulatory regions (e.g., promoters, transcriptional regulatory regions, and translational termination regions) and/or the DGAT1 polynucleotide may be native/analogous to the host cell or to each other. Alternatively, the regulatory regions and/or the modified DGAT1 polynucleotide may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the modified DGAT1 polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

The promoter of the recombinant DNA constructs of the invention can be any type or class of promoter known in the art, such that any one of a number of promoters can be used to express the various modified DGAT1 sequences disclosed herein, including the native promoter of the polynucleotide sequence of interest. The promoters for use in the recombinant DNA constructs of the invention can be selected based on the desired outcome.

Provided are plants, plant cells, plant parts, seeds, and grain comprising at least one of the modified DGAT1 polynucleotide sequences or recombinant DNA constructs, described herein, so that the plants, plant cells, plant parts, seeds, and/or grain express any of the modified DGAT1 polypeptides described herein. In certain embodiments, the plants, plant cells, plant parts, seeds, and/or grain have stably incorporated at least one modified DGAT1 polynucleotide into its genome. In certain embodiments, the plants, plant cells, plant parts, seeds, and/or grain can comprise multiple modified DGAT1 polynucleotides (i.e., at least 1, 2, 3, 4, 5, 6 or more).

In certain embodiments, the modified DGAT1 polynucleotides in the plants, plant cells, plant parts, seeds, and/or grain are operably linked to a heterologous regulatory element, such as but not limited to a constitutive, tissue-preferred, or other promoter for expression in plants or a constitutive enhancer.

In certain embodiments, the plants, plant cells, plant parts, seeds, and grain comprise a targeted genetic modification that results in the plant, plant cell, plant part, seed, or grain encoding any of the modified DGAT1 polypeptides described herein. In certain embodiments, the targeted genetic modification is at a genomic locus that encodes an endogenous DGAT1 polypeptide wherein the introduced targeted genetic modification results in the genomic locus encoding any of the modified DGAT1 polypeptides described herein. For example, a modified DGAT1 polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 50, 67, 69, 71, 73, 75, 77, 79, 81, 83 or 85 and comprising a modification at one or more amino acid residues corresponding to position of H16, K45, F75, F106, S140, I164, R200, T210, Y225, D252, S258, V267, V293, I296, L358, A416, I473, or L477 of SEQ ID NO: 2.

As used herein, a “targeted” genetic modification or “targeted” DNA modification, refers to the direct manipulation of an organism's genes. The targeted modification may be introduced using any technique known in the art, such as, for example, plant breeding, genome editing, or single locus conversion.

The targeted genetic modification of the genomic locus may be introduced using any genome modification technique known in the art or described herein. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide modification template.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The plant of the composition and methods described herein is not particularly limited and may be any plant for which increased oil content in the seed is desired. Examples of plant species of interest include, but are not limited to, maize (Zea mays), Brassica sp. (e.g., B. napus, B. rapa, B. juncea), particularly those Brassica species useful as sources of seed oil, alfalfa (Medicago sativa), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), peanuts (Arachis hypogaea), cotton (Gossypium barbadense, Gossypium hirsutum), coconut (Cocos nucifera), olive (Olea europaea), cashew (Anacardium occidentale), macadamia (Macadamia integrifolia), almond (Prunus amygdalus), green beans (Phaseolus vulgaris), and lima beans (Phaseolus limensis), peas (Lathyrus spp).

In certain embodiments, plants of the present disclosure are oil-seed plants (e.g., cotton, soybean, safflower, sunflower, Brassica, maize, alfalfa, palm, coconut, etc.) and/or leguminous plants (e.g., beans and peas; beans include guar, locust bean, fenugreek, soybean, garden beans, cowpea, mungbean, lima bean, fava bean, lentils, chickpea). In certain embodiments, soybean, sunflower, and/or Brassica plants are optimal, and in yet other embodiments soybean plants are optimal.

For example, in certain embodiments, soybean plants are provided that comprise, in their genome, a polynucleotide that encodes a DGAT1 polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprising a modification at one or more amino acid residues corresponding to position of H16, K45, F75, F106, S140, I164, R200, T210, Y225, D252, 5258, V267, V293, I296, L358, A416, I473, or L477, or any combination thereof, of SEQ ID NO: 2.

For example, in certain embodiments, soybean plants are provided that comprise, in their genome, a polynucleotide that encodes a DGAT1 polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50 and comprising a modification at one or more amino acid residues corresponding to position H16, K48, F81, F112, S146, I170, R206, T216, Y231, D258, S264, V273, V299, I302, L364, A422, I479, or L483, or any combination thereof, of SEQ ID NO: 50.

In certain embodiments, the plants described herein are elite plant lines (e.g., elite soybean line). In certain embodiments, the plant cells, plant parts, seeds, and grain are isolated from or produced by an elite plant line. As used herein, “elite line” refers to any line that has resulted from breeding and selection for superior agronomic performance that allows a producer to harvest a product of commercial significance. Numerous elite lines are available and known to those of skill in the art of plant breeding (e.g., soybean, canola, and sunflower breeding). An “elite population” is an assortment of elite individuals or lines that can be used to represent the state of the art in terms of agronomically superior genotypes of a given crop species, such as soybean.

In certain embodiments, the plants, plant cells, plant parts, seeds, and grain described herein have increased fatty acid or oil content when compared to a control plant, plant cell, plant part, seed, or grain (e.g., plant, plant cell, plant part, seed, or grain comprising a comparable polynucleotide which lacks the modification). In certain embodiments, the fatty acid or oil content in the plant, plant cell, plant part, seed, or grain containing or expressing the modified DGAT1 polynucleotide or polypeptide disclosed herein comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% increase as compared to the fatty acid or oil content of the control plant, plant cell, plant part, seed, or grain. In certain embodiments, the fatty acid or oil content is increased in a seed containing or expressing the modified DGAT1 polynucleotides or polypeptides. In certain embodiments, the seed is a soybean seed. In certain embodiments, the fatty acid or oil content is increased in the leaf tissue of a plant containing or expressing the modified DGAT1 polynucleotides or polypeptides. In certain embodiments, the leaf tissue is from a soybean plant. In certain embodiments, the plant, plant cell, plant part, seed, or grain (e.g., soybean seed) have an increased oil or fatty acid content as described herein, and optionally modified amounts of fatty acids, such as at least a 5%, 10%, 15%, 20%, 25%, 30%, 40%, or 50% increase in oleic acid content expressed by weight as a proportion of the total fatty acid content as described herein.

In certain embodiments, the plant, plant cell, plant part, seed, or grain containing or expressing the modified DGAT1 polynucleotides or polypeptides comprises least a 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, or 8.0 percentage points increase and less than about 10.0, 9.5, 9.0, 8.5, 8.0, 7.5, 7.0, 6.5, 6.0, 5.5, 5.0 or 4.5 percentage points increase in fatty acid or oil content measured on a dry weight basis relative to a control. In certain embodiments, the fatty acid or oil content is increased in a seed containing or expressing the modified DGAT1 polynucleotides or polypeptides. In certain embodiments, the seed is a soybean seed. In certain embodiments, the fatty acid or oil content is increased in the leaf tissue of a plant containing or expressing the modified DGAT1 polynucleotides or polypeptides. In certain embodiments, the leaf tissue is from a soybean plant.

As used herein, “percentage point” (pp) difference, change, increase or decrease refers to the arithmetic difference of two percentages, e.g. [transgenic or genetically modified value (%) −control value (%)]=percentage points. For example, a modified seed may contain 20% by weight of a component and the corresponding unmodified control seed may contain 15% by weight of that component. The difference in the component between the control and transgenic seed would be expressed as 5 percentage points.

In certain embodiments, the plant, plant cell, plant part, seed, or grain containing or expressing the modified DGAT1 polynucleotides or polypeptides comprises or further comprises at least a 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.5%, 4.0%, 4.5% or 5.0% increase in protein content relative to the protein content of a plant, plant cell, plant part, seed, or grain expressing the polypeptide without the modifications. The increase in protein content in the cell or seed can be an increase of at least at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9 or 2.0 percentage points and less than about 5.0, 4.5, 4.0, 3.5, 3.0, 2.5, 2.0, or 1.5 percentage points by weight of the cell relative to control.

In certain embodiments, the plants containing or expressing the modified DGAT1 polynucleotides or polypeptides have a yield that is greater than or within 0.5%, 1%, 1.5%, 2%, 2.5%, 3%, 3.5%, 4%, 4.5%, or 5%, as compared to the corresponding control plant, for example, one which has a similar genetic background but lacks the introduced modifications.

In certain embodiments, the plants described herein (e.g., plants comprising a modified polynucleotide encoding a modified DGAT1 polypeptide) have a yield of soybean seeds by weight at 13% moisture that is at least 80%, 81%, 82%, 83%, 84%, 85%, 86%, 87%, 88%, 89%, 90%, 91%, 92%, 93%, 94%, 95%, 96%, 97%, 98% 99%, 100%, 101%, 102%, 103%, 104%, 105%, 106%, 107%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134% or 135% and less than 250%, 240%, 203%, 220%, 210%, 200%, 195%, 190%, 185%, 180%, 175%, 170%, 165%, 160%, 155%, 150%, 145% or 140% of the yield of seeds by weight of soybean variety 93B83 (U.S. Pat. No. 5,792,909), when grown under the same environmental conditions. Representative seed of soybean variety 93B83 were deposited under ATCC Accession No. 209766 on Apr. 10, 1998. As used herein, “under the same environmental conditions” means the plants are grown in proximity in the field or a greenhouse under non-stress conditions suitable for growth of a soybean plant to maturity, with the plants being exposed to the same environment and seeds harvested from each plant at maturity growth stage R8.

Applicant has made a deposit of at least 2500 seeds of Soybean Variety 93B83 with the American Type Culture Collection (ATCC), 10801 University Boulevard, Manassas, Va. 20110 USA, as ATCC Deposit No. 209766. The seeds were deposited with the ATCC on Apr. 10, 1998 have been accepted under the Budapest Treaty. This deposit of the Soybean Variety 93B83 will be maintained in the ATCC depository, which is a public depository, for a period of 30 years, or 5 years after the most recent request, or for the effective life of the patent, whichever is longer, and will be replaced if it becomes nonviable during that period. Additionally, Applicant has satisfied all the requirements of 37 C.F.R.§§ 1.801-1.809. Upon allowance of any claims in the application, the Applicant(s) will maintain and will make this deposit available to the public pursuant to the Budapest Treaty.

As used herein, “yield” refers to the amount of agricultural production harvested per unit of land and may include reference to bushels per acre or kilograms per hectare of a crop at harvest, as adjusted for grain moisture. Grain moisture is measured in the grain at harvest. The adjusted test weight of grain is determined to be the weight in pounds per bushel or kilogram, adjusted for grain moisture level at harvest.

In certain embodiments, the plants containing or expressing the modified DGAT1 polynucleotides or polypeptides have a protein content that is greater than or within 0.5%, 1%, 1.5%, 2%, 2.5%, 3%, 3.5%, 4%, 4.5%, 5%, or 10% as compared to the corresponding control plant, for example, one which has a similar genetic background but lacks the introduced modifications.

Any plant having an inventive polynucleotide sequence disclosed herein can be used to make a food or a feed product. Such methods comprise obtaining a plant, explant, seed, plant cell, or cell comprising the polynucleotide sequence and processing the plant, explant, seed, plant cell, or cell to produce a food or feed product.

In certain embodiments, the seeds (e.g., soybean seeds) comprising or expressing the modified DGAT1 polypeptides described herein can be processed to produce oil and protein. Methods of processing the soybean seeds to produce oil and protein are provided which include one or more steps of dehulling the seeds, crushing the seeds, heating the seeds, such as with steam, extracting the oil, roasting, and extrusion. Processing and oil extraction can be done using solvents or mechanical extraction. In plant cells, seeds, soybean cells and soybean seeds expressing the modified polynucleotides, the oil content can be increased by at least 5%, 10%, 15% or 20% compared to a comparable cell or seed expressing the polynucleotide without the modification.

Products formed following processing include, without limitation, soy nuts, soy milk, tofu, texturized soy protein, soybean oil, soy protein flakes, isolated soy protein. Crude or partially degummed oil can be further processed by one or more of degumming, alkali treatment, silica absorption, vacuum bleaching, hydrogenation, interesterification, filtration, deodorization, physical refining, refractionation, and optional blending to produce refined bleached deodorized (RBD) oil.

The oil and protein can be used in animal feed and in food products for human consumption. Provided are food products and animal feed comprising oils, protein and compositions and described herein which contain or are derived from the modified polynucleotides and modified polypeptides. The food products and animal feed may comprise nucleotides comprising one or more of the modified alleles disclosed herein and the modified polynucleotides, polypeptides and plant cell disclosed herein.

In some embodiments, the modified DGAT1 polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide. In certain embodiments, the molecular stack comprises at least one polynucleotide that confers tolerance to glyphosate, at least one polynucleotide that confers tolerance to 2,4-D, and at least one polynucleotide that confers tolerance to glufosinate.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”).

The plant, plant cell, plant part, seed, and/or grain comprising a polynucleotide sequence disclosed herein can also be combined with at least one other trait to produce plants that further comprise a variety of desired trait combinations. For instance, the plant, plant cell, plant part, seed, and/or grain having the polynucleotide sequence may be stacked with polynucleotides encoding polypeptides having pesticidal and/or insecticidal activity, or a plant, plant cell, plant part, seed, and/or grain comprising a polynucleotide sequence provided herein may be combined with a plant disease resistance gene.

In certain embodiments, the molecular stack comprises at least one additional polynucleotide that confers increased seed protein or oil content.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Provided are methods for increasing seed oil content comprising expressing in a plant a modified polynucleotide encoding any of the modified DGAT1 polypeptides described herein.

In certain embodiments, the method comprises: expressing in a plant cell (e.g., regenerable plant cell) a recombinant DNA construct comprising a polynucleotide described herein; and generating the plant from the plant cell. In certain embodiments, the polynucleotide is operably linked to at least one regulatory sequence. In certain embodiments, the at least one regulatory sequence is a heterologous promoter. The recombinant DNA construct for use in the method may be any recombinant DNA construct provided herein. In certain embodiments the recombinant DNA is expressed by introducing into a plant, plant cell, plant part, seed, and/or grain the recombinant DNA construct, whereby the polypeptide is expressed in the plant, plant cell, plant part, seed, and/or grain. In certain embodiments the recombinant DNA construct is incorporated into the genome of the plant.

Various methods can be used to introduce the modified DGAT1 sequence or recombinant DNA comprising the modified DGAT1 sequence into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

Stable transformation is intended to mean that the polynucleotide introduced into a plant integrates into the genome of the plant of interest and is capable of being inherited by the progeny thereof. “Transient transformation” is intended to mean that a polynucleotide is introduced into the plant of interest and does not integrate into the genome of the plant or organism or a polypeptide is introduced into a plant or organism.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the modified DGAT1 sequences can be provided to a plant using a variety of transient transformation methods. Such transient transformation methods include, but are not limited to, the introduction of the modified DGAT1 protein directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide disclosed herein can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided, and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

One of skill will recognize that after the expression cassette containing the inventive polynucleotide is stably incorporated in transgenic plants and confirmed to be operable, it can be introduced into other plants by sexual crossing. Any of a number of standard breeding techniques can be used, depending upon the species to be crossed.

Parts obtained from the regenerated plants described herein, such as flowers, seeds, leaves, branches, fruit, and the like are included, provided that these parts comprise cells comprising the inventive polynucleotide. Progeny and variants, and mutants of the regenerated plants are also included, provided that these parts comprise the introduced nucleic acid sequences.

In one embodiment, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

In certain embodiments, the method comprises: modifying an endogenous gene (e.g., endogenous DGAT1 gene) in a plant to encode any of the modified DGAT1 polypeptides described herein. In certain embodiments, the method comprises introducing into a plant cell (e.g., regenerable plant cell) a targeted genetic modification of an endogenous DGAT1 gene to produce any of the modified DGAT1 polypeptides described herein and generating a plant from the plant cell. In certain embodiments, the endogenous DGAT1 gene comprises a polynucleotide sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of any one of SEQ ID NOs: 1, 49, 66, 68, 70, 72, 74, 76, 78, 80, 82, or 84. In certain embodiments, the endogenous DGAT1 gene encodes a polypeptide comprising an amino acid sequence that is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of any one of SEQ ID NOs: 2, 50, 67, 69, 71, 73, 75, 77, 79, 81, 83, or 85.

In certain embodiments, the method comprises providing a guide RNA, at least one polynucleotide modification template, and at least one Cas endonuclease to a plant cell, wherein the at least one Cas endonuclease introduces a double stranded break at an endogenous DGAT1 gene in the plant cell and generates any of the modified polynucleotides described herein, obtaining a plant from the plant cell; and generating a progeny plant that comprises the polynucleotide and produces seeds having an increased oil content as compared to a control plant not comprising the polynucleotide.

Various methods can be used to introduce a genetic modification at a genomic locus that encodes a DGAT1 polypeptide into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide modification template.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015/0082478, WO2015/026886, WO2016007347, and WO201625131, all of which are incorporated by reference herein.

In certain embodiments the genetic modification is introduced without introducing a double strand break using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

In certain embodiments, the plant generated from the methods described herein produce seeds that have an increase in total oil content when compared to a seed or plant comprising a comparable polynucleotide which lacks the modification.

In certain embodiments, the oil content in the seeds of the plants produced by the methods described herein comprise an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45% or 50% relative to the oil content of a seed expressing the polypeptide without the modifications. In certain embodiments, the seed has modified amounts of fatty acids, such as at least a 5%, 10%, 15%, 20%, 25%, 30%, 40%, or 50% increase in oleic acid content expressed by weight as a proportion of the total fatty acid content as described herein. In certain embodiments, the seed comprises least a 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, or 8.0 percentage points increase and less than about 10.0, 9.5, 9.0, 8.5, 8.0, 7.5, 7.0, 6.5, 6.0, 5.5, 5.0 or 4.5 percentage points increase in oil content measured on a dry weight basis relative to a control seed (e.g., seed comprising a non-modified polypeptide).

In certain embodiments, the plants generated from the methods described herein have a yield that is greater than or within 0.5%, 1%, 1.5%, 2%, 2.5%, 3%, 3.5%, 4%, 4.5%, or 5%, as compared to the corresponding control plant, for example, one which has a similar genetic background but lacks the introduced modifications.

In certain embodiments, the plants generated from the methods described herein have a protein content that is greater than or within 0.5%, 1%, 1.5%, 2%, 2.5%, 3%, 3.5%, 4%, 4.5%, 5%, or 10% as compared to the corresponding control plant, for example, one which has a similar genetic background but lacks the introduced modifications

Further provided herein are methods of producing a seed having increased protein and/or oil content comprising crossing a first plant line comprising any of the modified polynucleotides described herein with a second plant line and harvesting the seed produced thereby. In certain embodiments, the second plant line does not comprise the modified polynucleotide. In certain embodiments, the method further comprises the step of backcrossing a second-generation progeny plant that comprises the polynucleotide to the parent plant that lacks the polynucleotide, thereby producing a backcross progeny plant that produces seed with increased oil content.

As used herein, “progeny” comprises any subsequent generation of a plant, and can include F1 progeny, F2 progeny F3 progeny and so on.

Also provided is a method of screening for the presence or absence of the modified DGAT1 polynucleotide encoding any of the modified DGAT1 polypeptides described herein in a plurality of genomic soybean DNA samples. Methods of extracting modified DNA from a sample or detecting the presence of DNA corresponding to the modified genomic sequences comprising deletions or substitutions disclosed herein in DGAT1 sequences are provided. Such methods comprise contacting a sample comprising soybean genomic DNA with a DNA primer set, that when used in a nucleic acid amplification reaction, such as the polymerase chain reaction (PCR), with genomic DNA extracted from soybeans produces an amplicon that is diagnostic for either the presence or absence of the modified polynucleotide or modified DGAT1 alleles. The methods include the steps of performing a nucleic acid amplification reaction, thereby producing the amplicon and detecting the amplicon. In some embodiments one of the pair of DNA molecules comprises the wild type sequence where the modification such as a deletion occurs with the second of the pair being upstream or downstream as appropriate and suitably in proximity to the wild type sequence where the modification such as deletion occurs, such that an amplicon is produced when the wild type allele is present, but no amplicon is produced when the modified allele is present.

Probes and primers are provided which are of sufficient nucleotide length to bind specifically to the target DNA sequence under the reaction or hybridization conditions. Suitable probes and primers are at least 10, 11, 12, 13, 14, 15, 16, 17, 18, 19, 20, 21, 22, 23, 24, 25, 26, 27, 28, 29 or 30 nucleotides in length, and less than 35, 34, 33, 32, 31, 30, 29, 28, 27, 26, 2,5 2,4 23, 22, 21, 20, 19, 18, 17, 16, 15, 14, 13, or 12 nucleotides in length. Such probes and primers can hybridize specifically to a target sequence under high stringency hybridization conditions. Preferably, probes and primers have complete or 100% DNA sequence similarity of contiguous nucleotides with the target sequence, although probes which differ from the target DNA sequence but retain the ability to hybridize to target DNA sequence may be also be used. Reverse complements of the primers and probes disclosed herein are also provided and can be used in the methods and compositions described herein.

In certain embodiments, the method comprises contacting a plurality of genomic soybean DNA samples, at least some of which comprise the polynucleotide, with a first DNA primer molecule and a second DNA primer molecule, performing the nucleic acid amplification, thereby producing a DNA amplicon molecule indicating the presence of the polynucleotide or a wild-type DGAT1 nucleotide sequence, and detecting the DNA amplicon molecules, wherein the presence, absence or size of the DNA amplicon molecule indicates the presence or absence of the polynucleotide in the at least one of the plurality of genomic soybean DNA samples. In certain embodiments, the method includes the steps of performing a nucleic acid amplification reaction, thereby producing the amplicon and detecting the amplicon. In some embodiments one of the pair of DNA molecules comprises the wild type sequence where the modification such as a deletion occurs with the second of the pair being upstream or downstream as appropriate and suitably in proximity to the wild type sequence where the modification such as deletion occurs, such that an amplicon is produced when the wild type allele is present, but no amplicon is produced when the modified allele is present.

The following are examples of specific embodiments of some aspects of the invention. The examples are offered for illustrative purposes only and are not intended to limit the scope of the invention in any way.

### Example 1

This example demonstrates the creation of a GmDGAT1a library of variants.

A library of variants from the Glycine max diacylglycerol acyltransferase 1a (GmDGAT1a; SEQ ID NO: 2) protein was created using recursive sequence recombination methods. Fifteen GmDGAT1a amino acid substitutions were targeted for this library: S140A, I164M, R200K, A215S, Y225F, D252E, V267L, V293L, I296V, I297V, L358V, A416S, V477I, L450V, and L477V. Additional diversity was also expected to be present in this library due to the random modifications that are inherent in this PCR-based method. The library was cloned into a shuttle vector made by modifying pRS246 (Stratagene) to include a 1.0 kb promoter and a 0.5 kb terminator from Saccharomyces cerevisiae 3-phosphoglycerate kinase (PGK1) to drive expression of the GmDGAT1a variants in yeast. The library was first transformed into E. coli and about 10,000 transformed colonies were plated. These colonies were pooled and total plasmid DNA was isolated and stored for subsequent transformation of yeast as needed. To assess the quality of the library, plasmid DNA from 17 independent E. coli colonies were prepared and the DNA sequences of the 17 random GmDGAT1a variants were determined to identify amino acid substitutions (Table 3) These 17 GmDGAT1a variants contained from one to five amino acid substitutions each. Of the 15 targeted substitutions, 11 were represented in at least one of these 17 variants, and 4 were not observed. The planned substitutions Y225F and V267L were the most frequently observed, with each being present in 7 of the 17 variants (41%). Unplanned substitutions were observed at 13 amino acid positions in these 17 variants. The results of this sequence analysis of random GmDGAT1a variants suggested that the library contained sufficient diversity to warrant screening in yeast to identify strains with high oil content.

TABLE 3
Amino acid substitutions present in 17 random variants from the GmDGAT1a library

| | Planned/Unplanned | | | | | | | | | | | | | | |
|---|---|---|---|---|---|---|---|---|---|---|---|---|---|---|---|
| | U | U | U | U | U | U | P | P | U | U | P | U | P | P | P |
| | GmDGAT1a position | | | | | | | | | | | | | | |
| | 5 | 14 | 27 | 49 | 64 | 111 | 140 | 164 | 169 | 174 | 200 | 211 | 215 | 225 | 252 |
| | WT GmDGAT1a aa | | | | | | | | | | | | | | |
| | D | L | G | S | Q | V | S | I | A | I | R | L | A | Y | D |
| random variant 1 | | P | | | | | | | | | | | | | |
| random variant 2 | | | | | | E | | | | | | | | | |
| random variant 3 | | | D | | | | | | | | K | | | | |
| random variant 4 | | | | | | | | | | T | | | | | |
| random variant 5 | | | | | R | | | | | | | | | F | |
| random variant 6 | | | | | | | | | | | | | | F | E |
| random variant 7 | | | | | | | A | | | | | | | | |
| random variant 8 | V | | | | | | A | | | | | | S | | |
| random variant 9 | | | | | | | | | | | | | | F | |
| random variant 10 | | | | P | | | | M | V | | | | | F | |
| random variant 11 | | | | | | | | | | | | S | | | E |
| random variant 12 | | | | | | | | | | | | | | F | |
| random variant 13 | | | | | | | A | | | | | | | | |
| random variant 14 | | | | | | | | | | | | | | F | |
| random variant 15 | | | | | | | | | | | | | | F | E |
| random variant 16 | | | | | | | A | | | | | | | | |
| random variant 17 | | | | | | | | | | | | | | | E |
| freq in 17 variants (%) | 6 | 6 | 6 | 6 | 6 | 6 | 24 | 6 | 6 | 6 | 6 | 6 | 6 | 41 | 24 |

| | Planned/Unplanned | | | | | | | | | | | | | |
|---|---|---|---|---|---|---|---|---|---|---|---|---|---|---|
| | U | P | P | P | P | U | P | U | P | U | P | P | P | |
| | GmDGAT1a position | | | | | | | | | | | | | |
| | 258 | 267 | 293 | 296 | 297 | 350 | 358 | 375 | 416 | 426 | 447 | 450 | 477 | total |
| | WT GmDGAT1a aa | | | | | | | | | | | | | aa |
| | S | V | V | I | I | F | L | N | A | L | V | V | L | subs |
| random variant 1 | | L | | | | | | | | | | | | 2 |
| random variant 2 | | | | | | | | | S | | | | V | 3 |
| random variant 3 | | L | | | | | V | | | | | | | 4 |
| random variant 4 | | | | | | | | | S | | | | V | 3 |
| random variant 5 | | | | | | | V | | | | | | | 3 |
| random variant 6 | | | | | | S | | | | | | | | 3 |
| random variant 7 | | L | | | | | | | | | | | | 2 |
| random variant 8 | | | | | | | V | | | | | | V | 5 |
| random variant 9 | G | L | | | | | V | | | | | | | 4 |
| random variant 10 | | | | | | | | | | | | | | 4 |
| random variant 11 | | | | | | | | S | | | | L | | 4 |
| random variant 12 | | L | | | | | | | | | | | | 2 |
| random variant 13 | | L | | | | | | | | P | | | | 3 |
| random variant 14 | | | | | | | V | | S | | | | | 3 |
| random variant 15 | | | | | | | | | | | | | | 2 |
| random variant 16 | | L | | | | | V | | | | | | | 3 |
| random variant 17 | | | | | | | | | | | | | | 1 |
| freq in 17 variants (%) | 6 | 41 | 0 | 0 | 0 | 6 | 35 | 6 | 18 | 6 | 0 | 6 | 18 | |

### Example 2

This example demonstrates the screening and isolation of GmDGAT1a high oil variants in yeast

The library of GmDGAT1a variants was transformed into a Saccharomyces cerevisiae strain deficient in the genes encoding DGAT and PDAT, such that the dga1/lro1 strain was incapable of producing high oil until transformed with an effective GmDGAT1a variant. The transformation procedure was accomplished using Yeastmaker™ Yeast Transformation System 2 kit (Takara) following manufacture's protocol. Heat shock treatment was performed at 39° C. for 45 min and 2 M DTT was added to the transformation solution to increase efficiency, as deviations from the protocol.

To screen for high oil strains, two low density enrichment steps were performed followed by staining with the lipophilic dye Nile Red, similar to a previously described method (Roesler et al., Plant Physiol, 171: 878-893 (2016)). About 60,000 transformed yeast colonies were plated on 13.5 cm diameter Petri plates with synthetic dropout minimal media lacking uracil (SD-URA). Four mL of resuspended transformed cells were applied to each plate and the plates were then incubated at 30° C. for 72 hrs. The resulting colonies were scraped together and pooled, and then 250 μl of the pooled yeast were used to start a 25 ml culture that was grown at 30° C. for 3 days. The culture was harvested and resuspended in 1 mL of SD-URA medium and layered onto a 2 mL cushion of 60% glycerol in a 15 mL culture tube. The tubes were centrifuged at 2500 g for 5 min, and the yeast in the upper portion of the glycerol cushion was used to start a second 25 mL culture that was grown for 3 days, followed by harvesting, resuspending, layering on 60% glycerol and centrifuging, as before. Yeast obtained from the upper portion of the glycerol cushion was then plated with the appropriate dilution to allow picking of individual colonies to start cultures for Nile red staining.

From the approximate 60,000 colonies transformed, 480 colonies enriched for low-density were screened by Nile red fluorescence. Single colonies were grown in 4 mL of SD-URA medium for 72 hours at 30° C. Optical Density at 600 nm (OD₆₀₀) was measured and cultures were adjusted to approximately 1.0. In 96-well plates, cultures were diluted (1:10) in 180 μL of SD-URA medium and 2 μL of Nile red (0.05 mg/mL in acetone) was added to each well. Excitation/emission (489/581 nm) were measured using a fluorometer for 5 minutes. The Nile red fluorescence at the 3-minute time point was used to calculate the final value. Results are represented as Mean Nile Red Fluorescence/OD₆₀₀=(Nile red florescence of sample−Nile red fluorescence of blank)/(OD₆₀₀ of sample−OD₆₀₀ of SD-URA blank).

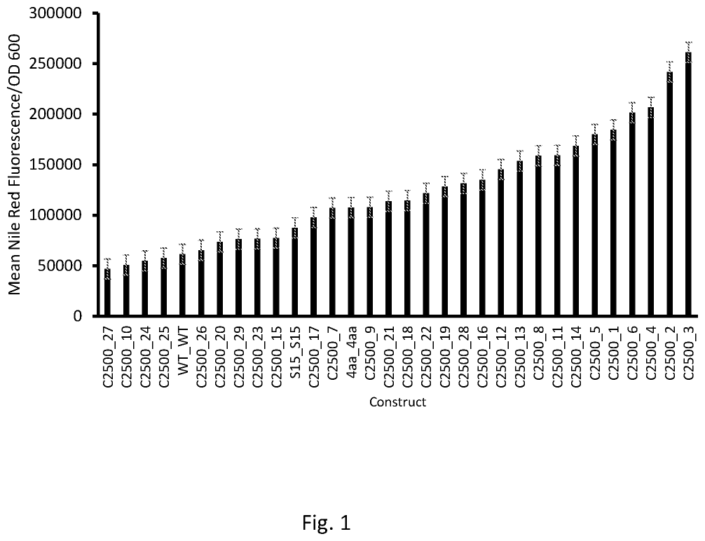

From the 480 colonies initially screened, five to seven colonies from each plate with the highest mean Nile red fluorescence/OD₆₀₀ were selected for a re-evaluation. For the re-evaluation test, the plasmid from each colony was recovered using the Zymoprep Yeast Plasmid Miniprep II kit. The amount of plasmid was increased in One Shot TOP10 Chemically Competent E. coli and re-transformed back into dga1/lro1 yeast. Three replicates of each colony were used to calculate mean Nile Red Fluorescence/OD₆₀₀ of these 29 constructs are shown in FIG. 1 in increasing value order. Wild type (WT; SEQ ID NO: 2) and a positive control with 4 amino acid substitutions (SEQ ID NO: 4) are also shown. Of the total 29 colonies re-screened, the top 11 colonies were advanced to in planta validation by N. benthamiana leaf infiltration. These 11 colonies had 137% to 326% increase in mean Nile Red Fluorescence/OD₆₀₀ compared to the WT (FIG. 1).

### Example 3

This example demonstrates the identification of the amino acid sequences of the GmDGAT1a variants.

To determine the variation in the DGAT1a protein resulting in high Nile Red fluorescence, plasmid from the yeast colonies were sequenced. Plasmids used for yeast transformations in the re-evaluation test (described in Example 2) were sent for sequencing at Elim Bioscience. Plasmids were submitted at 50 ng/μL per Elim Bioscience suggestion. Three primers were used to obtain full coverage with redundancy: 1. flanking forward primer: KF-Sc-FP1 (SEQ ID NO: 55), 2. flanking reverse primer: KF-Sc-RP2 (SEQ ID NO: 56), and 3. internal gene specific primer DG1a-FP1 (SEQ ID NO: 57).

Sequence analysis was completed using Vector NTI and Align X. Four of DGAT1a variants sequenced contained two to eight amino acid substitutions and all four variants contained the L477V modification (Table 4). Amino acid substitutions were validated by resequencing the plasmids that were utilized in the in planta validation by N. benthamiana leaf infiltration (see Example 5).

TABLE 4
Amino acid changes from yeast containing DGAT1a variants from selected strains with high Nile Red Fluorescence

| Nile | | | | | | | | | | | | | | | | | | | | |
|---|---|---|---|---|---|---|---|---|---|---|---|---|---|---|---|---|---|---|---|---|
| Red | Gene | Amino Acid Position | | | | | | | | | | | | | | | | | | |
| Rank | Variant | 16 | 45 | 75 | 106 | 114 | 140 | 164 | 192 | 200 | 206 | 225 | 252 | 258 | 267 | 293 | 296 | 358 | 416 | 477 |
| WT | GmDGAT1a | H | K | F | F | V | S | I | F | R | L | Y | D | S | V | V | I | L | A | L |

| | | | | | | | | | | | | | | | | |
|---|---|---|---|---|---|---|---|---|---|---|---|---|---|---|---|---|
| 2 | C2500_2 | L | | | | A | M | K | | | | | | | | V |
| 4 | C2500_6 | | E | | | | | | F | E | L | L | V | V | | V |
| 5 | C2500_1 | | | | | A | | | | | | | | | | V |
| 10 | C2500_13 | | | L | S | | | | | | | | | | S | V |

### Example 4

This example demonstrates the determination of GmDGAT1a specific activity and protein abundance in microsomal membranes.

Microsomal membrane preparations of yeast strains expressing GmDGAT1a variants were used for DGAT activity assays. Yeast cultures (typically 100 mL) were grown until early stationary phase, harvested by centrifugation at 1000×g for 5 min, and stored frozen at −80° C. Frozen pellets were thawed and resuspended in 4 ml of lysis buffer consisting of 50 mM Tris-HCl pH 8, 10 mM MgCl₂, 300 mM NaCl, 1 mM EDTA, 10% glycerol, and 1:100 Calbiochem Protease Inhibitor Cocktail Set III. The suspension was transferred to 2 mL tubes containing 0.5 mL of glass beads (425-600 μm diameter), and the cells were lysed by bead beating 3 times for 40 see each, at a setting of 4 m/s, using an MP Biomedicals FastPrep-24 instrument. Cooling on ice was done for 1 min between beatings. The lysate was centrifuged at 1500×g for 15 min at 4° C. The supernatant was then transferred to ultracentrifuge tubes and centrifuged at 100,000×g for 1 h 15 min at 6° C. The supernatant was discarded, and the microsomal membrane pellet was resuspended in 500 μL of 50 mM Hepes-NaOH pH 7.4, 10% glycerol. Pipetting was repeatedly done until the microsomal membrane suspension was homogeneous. The microsomal membrane suspension was aliquoted in 20 μL volumes into multiple tubes, frozen in liquid nitrogen, and stored at −80° C. Protein concentration was determined with the Coomassie Plus Protein Assay Reagent (Thermoscientific) using bovine serum albumen as the standard.

DGAT activity assays were performed using a method adapted from Caldo et al. (Plant Physiology, 175: 667-680 (2017)). The assays were done for 30 s at 25° C. in a 60 μL reaction volume containing 100 mM Hepes-NaOH pH 7.4, microsomal protein, 200 μM DAG (1,2-Dioleoyl-sn-glycerol) added from a 30 mM stock in ethanol, and 0.8 μM ¹⁴C-labeled oleoyl-CoA (American Radiolabeled Chemicals). Microsomal protein was added to the buffer, then DAG was added, and then the reaction was initiated by the addition of the ¹⁴C-oleoyl-CoA. The reaction was quenched with 10 μL of 10% SDS and vortexed for 4 s. Silica gel 60 matrix thin layer chromatography (TLC) plates (Merck) were pre-spotted with 2 μl of unlabeled TAG (glycerol trioleate) in 100 μL hexane to aid in later detection. The completed DGAT assay volume was co-spotted onto the TLC plates on top of the unlabeled TAG and the plates were dried for 2 h prior to performing the TLC with 80:20:1 hexane:diethyl ether:acetic acid. The triacylglycerol spots were visualized by iodine vapor staining, marked with a pencil, scraped off and transferred to scintillation vials. The ¹⁴C that had been incorporated into TAG was quantified by scintillation counting.

Protein abundance of GmDGAT1a variants in microsomal membranes, relative to GmDGAT1a (SEQ ID NO: 2), was determined by preparing immunoblots and probing them with rabbit polyclonal antibodies prepared against the peptide LRRRPSATSTAGLFNSPE (SEQ ID NO: 58). Microsomal membrane protein samples were prepared with NuPAGE LDS 4× sample buffer and 25 mM TCEP and heated for 10 min at 70° C. SDS-PAGE was performed using NuPage 10% BisTris gels with NuPAGE MOPS running buffer. Protein was transferred to nitrocellulose membranes using an Invitrogen I blot dry transfer system. The membranes were blocked for 30 min with 5% nonfat dry milk (NFDM) in PBST at room temperature, and then incubated at 4° C. overnight in PBST containing 1% NFDM and a 1:10,000 dilution of primary antisera. The membranes were washed 3 times for 10 min each in PBST at room temperature, and then incubated for 1 h at room temperature in PBST containing 1% NFDM and a 1:10,000 dilution of the secondary antibody, which was goat anti-rabbit H&L-RP conjugate. The membranes were again washed three times for 10 min each, then incubated 5 min with the Supersignal West Dura substrate. The blots were imaged with a GE Image Quant LAS4000 Luminescent Image Analyzer, and the DGAT bands were quantitated using Image Quant TL software. A standard curve of several concentrations of microsomes containing GmDGAT1a was included with each blot, which allowed calculation of the abundance of GmDGAT1a variants relative to GmDGAT1a. Results from four to five 5 independent immunoblots were averaged to determine the protein abundance of each GmDGAT1a variant.

The results of the DGAT immunoblots and activity assays are presented in Table 5. Most of the GmDGAT1a variants had substantially less DGAT protein in microsomal membrane preparations compared with the wild-type GmDGAT1a protein (Table 5). Less DGAT protein may be due to decreased expression or stability of the variants, or alternatively it may be because a larger proportion of the DGAT protein was in the large fat pads relative to the proportion in the microsomal membrane pellets, in the high oil yeast strains expressing the GmDGAT1a variants. After adjusting for DGAT protein abundance, all eight of the GmDGAT1a variants examined had significantly increased DGAT specific activity compared with wild type GmDGAT1a (Table 5).

TABLE 5
Protein abundance and specific activity of GmDGAT1a variants

| | DGAT protein | Specific | Specific activity |
|---|---|---|---|
| | abundance in | activity | adjusted for DGAT |
| | microsomes | (nmol/min/ | protein |
| | (relative | mg | (nmol/min/mg |
| | to | microsomal | microsomal |
| GmDGAT1a variant | GmDGAT1a) | protein)\hat{ } | protein)\hat{ } |
| GmDGAT1a (wild type) | 1 | 8.91 ± 0.58 | 8.91 ± 0.58 |
| GmDGAT1a (F75L, F106S, A416S, | 0.099 | 3.78 ± 0.078 | 38.2** ± 0.79 |
| L477V) | | | |
| GmDGAT1a (K45E, Y225F, D252E, | 0.092 | 2.45 ± 0.11 | 26.6** ± 1.1 |
| V267L, V293L, 1296V, L358V, | | | |
| L477V) | | | |
| GmDGAT1a (H16L, S140A, I164M, | 0.22 | 4.97 ± 0.53 | 22.6** ± 2.4 |
| R200K, L477V) | | | |
| GmDGAT1a (F106S, A416S, L477V) | 0.42 | 9.21 ± 0.21 | 21.9** ± 0.50 |
| GmDGAT1a (F106S, L477V) | 0.21 | 4.36 ± 0.10 | 20.8** ± 0.49 |
| GmDGAT1a (L477V) | 0.92 | 13.8 ± 0.76 | 15.0** ± 0.83 |
| GmDGAT1a (F106S) | 0.48 | 6.35 ± 0.19 | 13.2** ± 0.39 |
| GmDGAT1a (S140A, L477V) | 0.71 | 8.61 ± 0.52 | 12.1* ± 0.73 |

\hat{ }mean ± SE, n = 3
**significantly different from GmDGAT1a in t-test, p < 0.01
*significantly different from GmDGAT1a in t-test, p < 0.05

### Example 5

This example demonstrates the confirmation of high oil GmDGAT1a variants in N. benthamiana leaves.

GmDGAT1a variants were evaluated by transient expression in N. benthamiana leaves. Gene variants were subcloned into a binary vector between the GM-UBQ promoter and UBQ14 terminator using NEBuilder HiFi DNA Assembly. The binary vectors were transformed into Agrobacterium strain AGL1 by electroporation. Liquid cultures were inoculated from a preculture to a starting OD₆₀₀ of 0.01 and grown in a shaking incubator overnight at 28° C. Cells were pelleted and resuspended in infiltration buffer (5 mM MgS04, 5 mM MES (pH 5.6), and 150 μM acetosyringone). The cell suspensions were diluted to a final OD₆₀₀ of 0.07 prior to infiltration. N. benthamiana plants were grown in a greenhouse for three weeks and then transferred to a growth chamber set to 16-hour photoperiod, 24° C./20° C. light/dark temperature, and 65% relative humidity until the 6^{th} true leaf was fully expanded. The 6^{th} leaf was selected for infiltration (one leaf per plant). Agrobacterium suspensions were injected into the underside of leaves using a syringe without a needle while applying counter pressure. Plants were returned to the growth chamber for three days. Leaf discs were then sampled from infiltrated leaves, frozen and lyophilized prior to oil analysis.

Neutral lipids were extracted from −10 mg dried leaf discs four times with 100% hexane prior to analysis by HPLC-ELSD or GC-FID. For the HPLC-ELSD method, extracts were filtered through a PTFE 0.2 μm filter plate. Samples were then dried down and resuspended in 80 μL of heptane. A cyanopropyl column (Luna 5 μM CN 100A 250×4.6 mm; Phenomenx) was used to separate lipid species on an HPLC-ELSD with hexane as mobile phase A and methyl tertiary-butyl ether (MTBE):isopropanol (95:5 v/v) plus 0.2% acetic acid as mobile phase B, with a gradient of 0% to 100% B, with re-equilibration of the column to 0% B. A standard curve of tri-C17 triacylglycerol (TAG) was run with each sample set to quantify total TAG as Oil Content (% of DW). For the GC-FID method, extracted neutral lipids plus an internal standard (0.02 mg tri-C17:0 TAG) were concentrated under a stream of nitrogen to a volume of approximately 0.5 mL. TAG was isolated by solid phase extraction (SPE) using 96-well aminopropyl SPE plates (Phenomenex). Columns were preconditioned with 1 mL of hexane prior to loading samples. Columns were washed with 1 mL of hexane. TAG was eluted with 1 mL of hexane:dichloromethane:chloroform (83:12:5 v/v/v). The TAG fraction was concentrated under nitrogen and resuspended in 180 μl heptane. Fatty acids were derivatized by the addition of 20 μl of approximately 0.25 M trimethylsulfonium hydroxide in methanol (Sigma-Aldrich), followed by GC-FID for quantification.

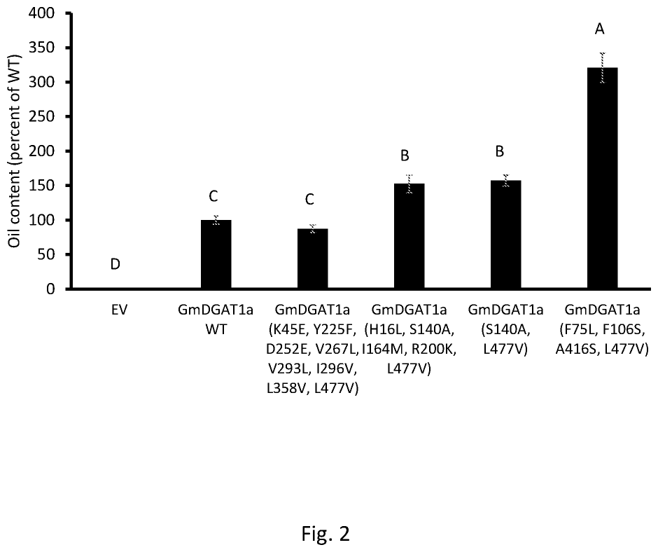

Total oil content was compared between experiments by normalizing to a percent of wild-type GmDGAT1a SEQ ID NO: 2 (FIG. 2). Three out of the four variants significantly increased oil content in N. benthamiana leaf compared to GmDGAT1a WT (SEQ ID NO: 2). One variant, GmDGAT1a (F75L, F106S, A416S, L477V; SEQ ID NO: 7), accumulated over 300% as much oil as GmDGAT1a WT.

### Example 6

This example demonstrates the identification of density gradient enriched modifications.

In order to further characterize the shuffled library, next generation sequencing was employed using Pacific Biosciences (PacBio) Sequel Instrument to compare the plasmid population before and after low-density enrichment. Low-density yeast cells isolated as described in Example 2 were used to inoculate an overnight liquid culture. Plasmid DNA was isolated from five aliquots of 5 mL culture using Yeast Plasmid Miniprep II kit (Zymoprep) with the following modifications. Cells were pelleted and resuspended in 600 μl Solution 1 plus 15 ul zymolase. Cell suspensions were incubated at 37° C. for four hours with occasional mixing, followed by one freeze-thaw cycle. 600 μL of Solution 2 was added to each tube and incubated at room temperature for five minutes. The solution was split into three microcentrifuge tubes (400 μL each) before adding 400 μL of Solution 3. Tubes were mixed and centrifuged at maximum speed for five minutes. Supernatant was transferred to a mini-prep column (Qiagen). The remainder of the protocol was completed according to Qiagen instructions and plasmids were eluted in 50 μL water. All elutions were pooled and digested with Exo V to remove genomic DNA. Exo V was heat inactivated at 70° C. for 30 minutes. Plasmid DNA was concentrated using DNA Clean & concentrator 5 (Zymoprep).

The post-density enrichment sample and the original plasmid library were sequenced by PacBio SMRT Sequencing, with long reads able to sequence the full gene in a single read. Reads were aligned globally using the Needle program from EMBOSS. Translated sequences were collapsed to calculate the frequency of sequences having the same substitutions. The dataset was filtered to compare sequences found in both the pre- and post-enrichment samples. Enrichment scores were normalized to account for total read number; variants enriched by greater than 3-fold are shown in Table 6. The frequency of individual planned substitutions across all variants was also calculated (Table 7).

The L477V substitution was prevalent among the most enriched variants (Table 6). The L477V substitution was also found in the most enriched sequence, which was 17-fold more prevalent in the post-enrichment sample compared to the pre-enrichment sample. The L477V (SEQ ID NO: 22) and the unplanned L477S single substitutions were among the most enriched variants, at 4.5 and 4.3-fold enriched respectively. Of the planned variants, the L477V substitution was enriched the most at 4.2-fold post-density gradient (Table 7).

TABLE 6
GmDGAT1a substitutions enriched post-density gradient

| Variants | Normalized fold enrichment |
|---|---|
| S140A, D252E, L477V | 17.0 |
| S140A, L477V (SEQ ID NO: 28) | 13.0 |
| S140A, V267L, L477V | 10.2 |
| S140A, R200K, L358V, L477V | 9.6 |
| S140A, L358V, L477V | 8.4 |
| D252E, L358V, L477V | 7.9 |
| S140A, R200K, V267L, L477V | 7.1 |
| S140A, R200K, L477V | 6.0 |
| I164M, L477V | 5.7 |
| V267L, L477V | 5.4 |
| L358V, L477V | 5.4 |
| Y225F, L477V | 4.8 |
| D252E, V267L, L477V | 4.8 |
| S140A, L358V, A416S, L477V | 4.5 |
| R200K, L358V, L477V | 4.5 |
| L477S | 4.5 |
| L477V (SEQ ID NO: 22) | 4.3 |
| S140A, R200K, L477S | 4.2 |
| S140A, Y225F, L477V | 4.2 |
| S140A, L477S | 4.2 |
| S140A, D252E, V267L, L358V, L477V | 4.0 |
| R200K, D252E, L477V | 4.0 |
| A416S, L477V (SEQ ID NO: 34) | 3.9 |
| L358V, A416S, L477V | 3.8 |
| S140A, A416S, L477V | 3.8 |
| S140A, D252E, V267L, L477V | 3.6 |
| S140A, A416S, L477S | 3.4 |
| R200K, V267L, L358V, L477V | 3.4 |
| S140A, D252E, L358V, L477V | 3.3 |
| R200K, L477V | 3.1 |
| V267L, A416S, L477V | 3.1 |

TABLE 7
GmDGAT1a planned substitutions enriched post-density gradient

| Planned | Normalized fold |
|---|---|
| substitutions | enrichment |
| S140A | 1.3 |
| I164M | 0.9 |
| R200K | 1.1 |
| A215S | 0.9 |
| Y225F | 0.5 |
| D252E | 1.0 |
| V267L | 0.7 |
| V293L | 1.0 |
| I296V | 1.0 |
| I297V | 1.3 |
| L358V | 0.7 |
| A416S | 0.9 |
| V447I | |
| V450L | 0.7 |
| L477V | 4.2 |

### Example 7

This example demonstrates that GmDGAT1a variants containing F106S increase oil content in N. benthamiana

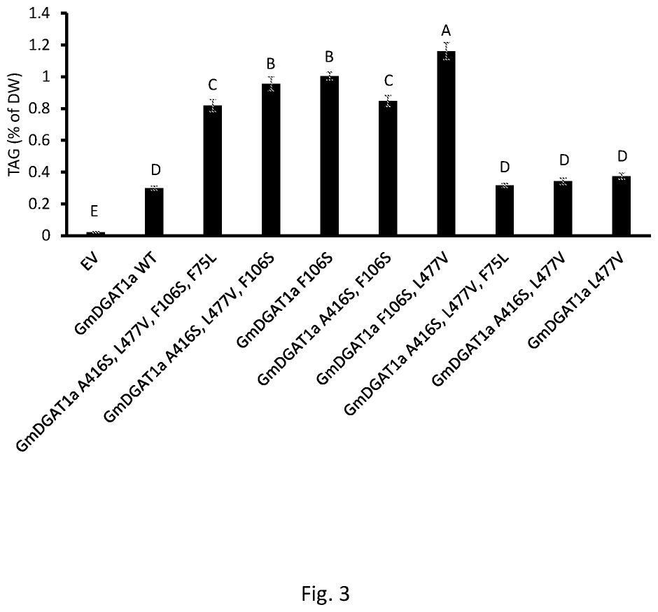

Additional variants with one to three amino acid substitutions were generated based on combinations of the variant GmDGAT1a F75L, F106S, A416S, L477V (SEQ ID NO: 7). These variants were evaluated in N. benthamiana leaf and oil was quantified with the GC-FID method described in Example 5. The results are presented in FIG. 3. All variants containing the F106S substitutions, including SEQ ID NO: 16, SEQ ID NO: 19, SEQ ID NO: 25, and SEQ ID NO: 30, increased oil content to the same level as GmDGAT1a F75L F106S A416S L477V (SEQ ID NO: 7) or higher. The F106S substitution alone (SEQ ID NO: 25) resulted in oil accumulation greater than 300% of GmDGAT1a WT SEQ ID NO: 2. The variant GmDGAT1a F106S L477V (SEQ ID NO: 19) accumulated nearly 400% of GmDGAT1a WT SEQ ID NO: 2 oil content. The L477V substitution in combination with F106S (SEQ ID NO: 19) resulted in oil levels that were significantly higher than any other variant tested. The L477V substitution was also identified through PacBio sequencing (see Example 6).

### Example 8

This example demonstrates additional amino acid substitutions at F106 increases total oil content in N. benthamiana

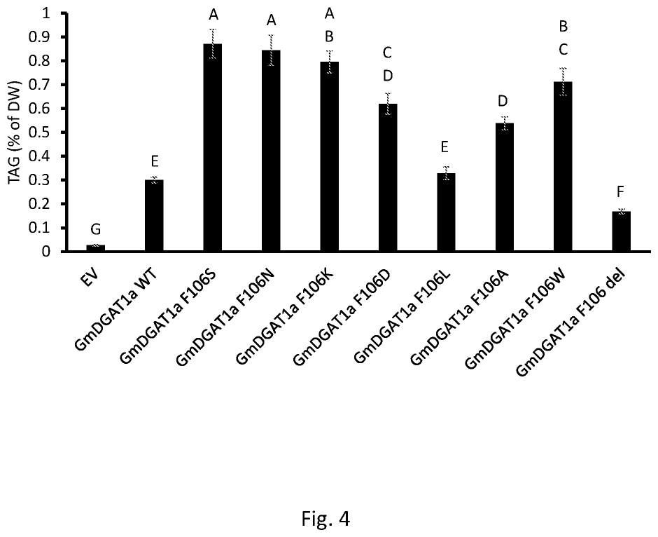

Additional constructs were designed to assess further substitutions at the F106 position (SEQ ID NO: 25). These variants were evaluated in N. benthamiana leaf and oil was quantified with the GC-FID method described in Example 5. FIG. 4 shows that substitutions F106S (SEQ ID NO: 25), F106N (SEQ ID NO: 36, F106K (SEQ ID NO: 38), F106D (SEQ ID NO: 40), F106A (SEQ ID NO: 44), and F106W (SEQ ID NO: 46), significantly increased oil accumulation compared to GmDGAT1a WT SEQ2. GmDGAT1a F106N (SEQ ID NO: 36) and GmDGAT1a F106K (SEQ ID NO: 38) accumulated TAG to levels that were statistically the same as the GmDGAT1a F106S (SEQ ID NO: 25) variant. Deletion of the F106 residue (SEQ ID NO: 48) significantly decreased TAG compared to WT.

### Example 9

This example demonstrates variants at the corresponding positions in GmDGAT1b in N. benthamiana increase oil content.

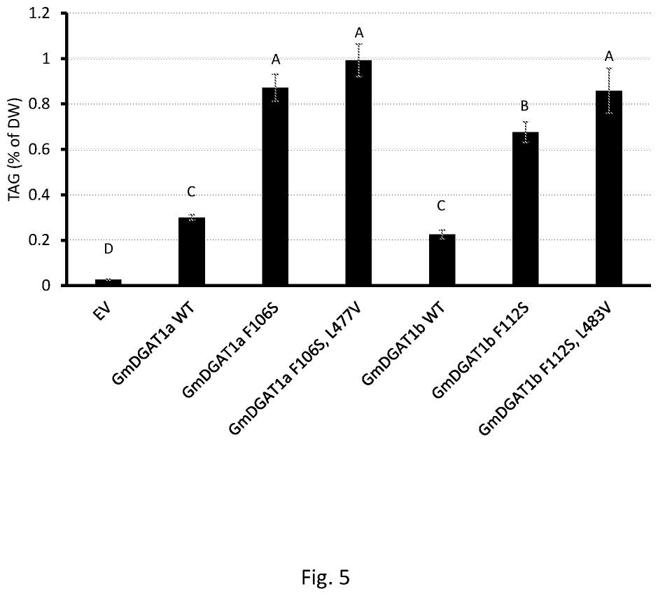

Variants were generated to assess substitutions in the homologous gene GmDGAT1b (SEQ ID NO: 50). Constructs were evaluated in N. benthamiana leaf and oil was quantified with the GC-FID method described in Example 5. FIG. 5 shows that variants GmDGAT1b F112S (SEQ ID NO: 52) and GmDGAT1b F112S L483V (SEQ ID NO: 54) increased oil to similar levels as the corresponding GmDGAT1a variants (SEQ ID NO: 25 and SEQ ID NO: 19).

### Example 10

This example demonstrates that the expression of the GmDGAT1a variants in soybean increase seed oil content.

The GmDGAT1a variants were cloned into an expression vector flanked by seed specific soybean β-conglycinin promoter and P. vulgaris phaseolin terminator. The binary expression vectors containing the GmDGAT1a variant cassettes (listed in Table 8) were transformed into soybean (Anand A, 2017). Transgenic Ti seed oil content was determined by single seed NIR, as previously described (Roesler et al., 2016). Since the Ti seed are segregating 3:1, there are large variances in the oil content. Overexpressing the GmDGAT1a WT (SEQ ID NO: 2) increases oil content. Overexpressing GmDGAT1a F75L F106S A416S L477V (SEQ ID NO: 7), GmDGAT1a F106S (SEQ ID NO: 25), GmDGAT1a F106S L477V (SEQ ID NO: 19), and GmDGAT1a L477V (SEQ ID NO: 22) also increase oil content compared to the greenhouse grown 93Y21 untransformed control. The variants GmDGAT1a F75L F106S A416S L477V (SEQ ID NO: 7), GmDGAT1a F106S (SEQ ID NO: 25), and GmDGAT1a L477V (SEQ ID NO: 22) significantly increase oil over the GmDGAT1a WT (SEQ ID NO: 2) (Table 8).

TABLE 8
Oil content of T₁ soybean seed over-expressing GmDGAT1a variants.

| | | | | Significant | Significant |
|---|---|---|---|---|---|
| | | Average | | increase | increase |
| | | Oil | Standard | compared | compared |
| Gene Variant | Number | % | Deviation | to WT | to 93Y21 |
| GmDGAT1a F75L F106S A416S | 249 | 20.86 | 2.60 | ** | ** |
| L477V (SEQ ID NO: 7) | | | | | |
| GmDGAT1a F106S (SEQ ID NO: | 229 | 20.61 | 2.63 | ** | ** |
| 25) | | | | | |
| GmDGAT1a F106S L477V (SEQ ID | 213 | 18.95 | 2.43 | | ** |
| NO: 19) | | | | | |
| GmDGAT1a L477V (SEQ ID NO: | 292 | 20.17 | 1.22 | ** | ** |
| 22) | | | | | |
| GmDGAT1a C2500_6 (SEQ ID NO: | 140 | 14.76 | 2.23 | | |
| 10) | | | | | |
| GmDGAT1a WT (SEQ ID NO: 2) | 252 | 19.71 | 1.30 | | ** |
| GH 93Y21 control | 147 | 18.42 | 1.30 | | |

**indicates significant increases, using t-test with p < 0.01 Average Oil % measured at 13% moisture

### Example 11

This example demonstrates the modification of endogenous DGAT1 genes to increase oil content in soybean.

To introduce expression of the DGAT1 protein variants described herein a polynucleotide modification template providing the information for editing the DGAT1 coding sequence will be created. Briefly, the polynucleotide modification template will comprise the nucleotide modifications that result in the amino acid changes between DNA fragments of the soybean DGAT1 genomic sequence used as homologous sequence for the DGAT1 gene editing.

Plasmids will also be designed for a single guide RNA (sgRNA) targeting the DGAT1 gene sequence and the Cas9 endonuclease from Streptococcus pyogenes. For example, a specific gRNA, GM-DGAT-CR6 (SEQ ID NO: 59), was designed to target the F106 region of the soybean DGAT1a gene (SEQ ID NO: 60). To facilitate delivery of the genome editing reagents into soybean cells and regeneration of plants, expression cassettes encoding a transformation selection marker and/or a cell division and callus growth-promoting protein may be used.

The gene editing plasmids will be co-delivered with the polynucleotide modification template to immature soybean embryos by known transformation techniques such as particle bombardment and/or agrobacterium or ochrobacterium mediated transformation. Transformed soybean embryos will be analyzed for insertion of the sequence encoding the DGAT1 protein variant using methods known in the art, such as PCR and sequencing.

Transformed soybean embryos comprising the DGAT1 protein variant will be generated into plants and oil content of the seeds and/or leaf tissue from the generated plants will be analyzed.

Plants comprising an endogenous gene modified to express the modified a DGAT1 protein variant are expected to have an increased oil content as compared to plants not comprising the modified endogenous DGAT1 gene.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A modified polynucleotide encoding a modified diacylglycerol acyltransferase-1 (DGAT1) polypeptide having at least 90% sequence identity to SEQ ID NO: 2, 50, 67, 69, 71, 73, 75, 77, 79, 81, 83 or 85 and comprising at least one modification selected from the group consisting of a non-phenylalanine at the position corresponding to position 106 of SEQ ID NO: 2, a non-histidine at the position corresponding to position 16 of SEQ ID NO: 2, a non-lysine at the position corresponding to position 45 of SEQ ID NO: 2, a non-phenylalanine at the position corresponding to position 75 of SEQ ID NO: 2, and a non-arginine at the position corresponding to position 200 of SEQ ID NO: 2, wherein when expressed in a plant cell, the polynucleotide increases the fatty acid content of the plant cell compared to a plant cell comprising a comparable polynucleotide without the modification.

2. The modified polynucleotide of claim 1, wherein the at least one modification comprises a modification selected from the group consisting of a leucine at the position corresponding to position 16 of SEQ ID NO: 2, a glutamic acid at the position corresponding to position 45 of SEQ ID NO: 2, a leucine at the position corresponding to position 75 of SEQ ID NO: 2, a serine at the position corresponding to position 106 of SEQ ID NO: 2, an asparagine at the position corresponding to position 106 of SEQ ID NO: 2, a lysine at the position corresponding to position 106 of SEQ ID NO: 2, an aspartic acid at the position corresponding to position 106 of SEQ ID NO: 2, a leucine at the position corresponding to position 106 of SEQ ID NO: 2, an alanine at the position corresponding to position 106 of SEQ ID NO: 2, a tryptophan at the position corresponding to position 106 of SEQ ID NO: 2, a deletion at the position corresponding to position 106 of SEQ ID NO: 2, and a lysine at the position corresponding to position 200 of SEQ ID NO: 2.

3. The modified polynucleotide of claim 1, wherein the encoded polypeptide further comprises at least one modification selected from the group consisting of a non-serine at the position corresponding to position 140 of SEQ ID NO: 2, a non-isoleucine at the position corresponding to position 164 of SEQ ID NO: 2, a non-threonine at the position corresponding to position 210 of SEQ ID NO: 2, a non-tyrosine at the position corresponding to position 225 of SEQ ID NO: 2, a non-aspartic acid at the position corresponding to position 252 of SEQ ID NO: 2, a non-serine at the position corresponding to position 258 of SEQ ID NO: 2, a non-valine at the position corresponding to position 267 of SEQ ID NO: 2, a non-valine at the position corresponding to position 293 of SEQ ID NO: 2, a non-isoleucine at the position corresponding to position 296 of SEQ ID NO: 2, a non-leucine at the position corresponding to position 358 of SEQ ID NO: 2, a non-alanine at the position corresponding to position 416 of SEQ ID NO: 2, a non-isoleucine at the position corresponding to position 473 of SEQ ID NO: 2 and a non-leucine at the position corresponding to position 477 of SEQ ID NO: 2.

4-23. (canceled)

24. The modified polynucleotide of claim 1, wherein the encoded modified DGAT1 polypeptide comprises at least one amino acid motif selected from the group consisting of APTLCYQ (SEQ ID NO: 61), FGDREFYXDWWNA (SEQ ID NO: 62) and LLYYHD (SEQ ID NO: 63), where X is any amino acid.

25-26. (canceled)

27. The modified polynucleotide of claim 1, wherein the encoded modified DGAT1 polypeptide comprises at least one amino acid motif selected from the group consisting of ESPLSSDX′IFX′QSHAGLX′NLCX′VVLX′AVNX′RLIIENLMKYGX′LI (SEQ ID NO 64) and GFIIEQYINPIVX′NSX′HPL (SEQ ID NO:65), wherein X′ is any amino acid.

28. (canceled)

29. The modified polynucleotide of claim 1, wherein the modified DGAT1 polypeptide further comprises a deletion of at least 1 and less than 101 amino acids, the deletion occurring in the N-terminal region of the DGAT1 polypeptide corresponding to the region at positions 1 to 101 of SEQ ID NO: 2, wherein the DGAT1 polypeptide has increased stability when expressed in a plant cell.

30. A recombinant DNA construct comprising a modified polynucleotide of claim 1.

31. (canceled)

32. A plant cell comprising the modified polynucleotide of claim 1, wherein the plant cell has an increased fatty acid content compared to a plant cell comprising a comparable DGAT1 polynucleotide without the modification.

33. The plant cell of claim 32, wherein the plant cell is a soybean cell.

34. (canceled)

35. A plant comprising the plant cell of claim 32.

36. (canceled)

37. A seed comprising the plant cell of claim 32, wherein the seed has an increased oil content compared to a seed comprising a comparable DGAT1 polynucleotide without the modification.

38. The seed of claim 37, wherein the seed is soybean seed.

39. The seed of claim 38, wherein the oil content in the seed is increased by at least 5% as compared to a control seed.

40. The seed of claim 39, wherein the seed further comprises a protein content that is greater than or within 5% as compared to a seed comprising the comparable polynucleotide without the modification.

41. (canceled)

42. A method of producing a plant having increased seed oil content, the method comprising:
a. expressing in the plant cell a modified polynucleotide encoding a modified diacylglycerol acyltransferase-1 (DGAT1) polypeptide having at least 90% sequence identity to SEQ ID NO: 2, 50, 67, 69, 71, 73, 75, 77, 79, 81, 83 or 85 and comprising at least one modification selected from the group consisting of a non-phenylalanine at the position corresponding to position 106 of SEQ ID NO: 2, a non-histidine at the position corresponding to position 16 of SEQ ID NO: 2, a non-lysine at the position corresponding to position 45 of SEQ ID NO: 2, a non-phenylalanine at the position corresponding to position 75 of SEQ ID NO: 2, and a non-arginine at the position corresponding to position 200 of SEQ ID NO: 2, and
b. generating a plant from the plant cell, wherein the plant produces seed having increased oil content compared to a control seed not comprising the modified polynucleotide.

43. The method of claim 42, wherein the method comprises introducing in the plant cell a recombinant DNA construct comprising the modified polynucleotide.

44. The method of claim 42, wherein the method comprises introducing into the plant cell a targeted genetic modification at an endogenous DGAT1 gene to produce the modified polynucleotide.

45. The method of claim 44, wherein the targeted genetic modification is introduced using enzyme selected from the group consisting of a polynucleotide-guided endonuclease, CRISPR-Cas endonucleases, base editing deaminases, a zinc finger nuclease, a transcription activator-like effector nuclease (TALEN), and engineered site-specific meganucleases.

46-49. (canceled)

50. The method of claim 42, wherein the plant cell is a soybean cell.

51. (canceled)

52. The method of claim 42, wherein the seed produced from the plant has at least a 5% increase in oil content as compared to the control seed.

53-59. (canceled)
